(19) United States
(12) Patent Application Publication
(10) Pub. No.: US 2009/0076275 A1
(43) Pub. Date: Mar. 19, 2009

(54) DIACYLGLYCEROL ACYLTRANSFERASE INHIBITORS

(76) Inventors: David Robert Bolin, Montclair, NJ (US); Jianping Cai, West Caldwell, NJ (US); Adrian Wai-Hing Cheung, Glen Rock, NJ (US); Robert Alan Goodnow, JR., Gillette, NJ (US); Matthew Michael Hamilton, Hackettstown, NJ (US); Shiming Li, Glastonbury, CT (US); Lee Apostle McDermott, Parlin, NJ (US); Weiya Yun, Warren, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/207,674

(22) Filed: Sep. 10, 2008

Related U.S. Application Data
(60) Provisional application No. 60/973,584, filed on Sep. 19, 2007.

(51) Int. Cl. C07D 413/12; C07D 417/12; C07D 471/04

(52) U.S. Cl. 546/119; 548/236; 548/200; 546/270.4; 546/199

(57) ABSTRACT

Provided herein are compounds of the formula (I):

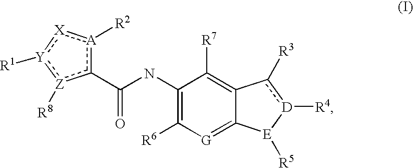

as well as pharmaceutically acceptable salts thereof, wherein the substituents are as those disclosed in the specification. These compounds, and the pharmaceutical compositions containing them, are useful for the treatment of diseases such as, for example, obesity, type II diabetes mellitus and metabolic syndrome.

### PRIORITY TO RELATED APPLICATION(S)

This application claims the benefit of U.S. Provisional Application No. 60/973,584 filed Sep. 19, 2007, which is hereby incorporated by reference in its entirety.

### FIELD OF THE INVENTION

The invention relates to inhibitors of diacylglycerol acyltransferase. The inhibitors are useful for the treatment of diseases such as obesity, type II diabetes mellitus, dyslipidemia and metabolic syndrome.

All documents cited or relied upon below are expressly incorporated herein by reference.

### BACKGROUND OF THE INVENTION

Triglycerides or triacylglycerols are the major form of energy storage in eukaryotic organisms. In mammals, these compounds are primarily synthesized in three tissues: the small intestine, liver, and adipocytes. Triglycerides or triacylglycerols support the major functions of dietary fat absorption, packaging of newly synthesized fatty acids and storage in fat tissue (see Subauste and Burant, Current Drug Targets—Immune, Endocrine & Metabolic Disorders (2003) 3, 263-270).

Diacylglycerol O-acyltransferase, also known as diglyceride acyltransferase or DGAT, is a key enzyme in triglyceride synthesis. DGAT catalyzes the final and rate-limiting step in triacylglycerol synthesis from 1,2-diacylglycerol (DAG) and long chain fatty acyl CoA as substrates. Thus, DGAT plays an essential role in the metabolism of cellular diacylglycerol and is critically important for triglyceride production and energy storage homeostasis (see Mayorek et al, European Journal of Biochemistry (1989) 182, 395-400).

DGAT has a specificity for sn-1,2 diacylglycerols and will accept a wide variety of fatty acyl chain lengths (see Farese et al, Current Opinions in Lipidology (2000) 11, 229-234). DGAT activity levels increase in fat cells as they differentiate in vitro and recent evidence suggests that DGAT may be regulated in adipose tissue post-transcriptionally (see Coleman et al, Journal of Molecular Biology (1978) 253, 7256-7261 and Yu et al, Journal of Molecular Biology (2002) 277, 50876-50884). DGAT activity is primarily expressed in the endoplasmic reticulum (see Colman, Methods in Enzymology (1992) 209, 98-104). In hepatocytes, DGAT activity has been shown to be expressed on both the cytosolic and luminal surfaces of the endoplasmic reticular membrane (see Owen et al, Biochemical Journal (1997) 323 (pt 1), 17-21 and Waterman et al, Journal of Lipid Research (2002) 43, 1555-156). In the liver, the regulation of triglyceride synthesis and partitioning, between retention as cytosolic droplets and secretion, is of primary importance in determining the rate of VLDL production (see Shelness and Sellers, Current Opinions in Lipidology (2001) 12,151-157 and Owen et al, Biochemical Journal (1997) 323 (pt 1), 17-21).

Two forms of DGAT have been cloned and are designated DGAT1 and DGAT2 (see Cases et al, Proceedings of the National Academy of Science, USA (1998) 95,13018-13023, Lardizabal et al, Journal of Biological Chemistry (2001) 276, 38862-38869 and Cases et al, Journal of Biological Chemistry (2001) 276, 38870-38876). Although both enzymes utilize the same substrates, there is no homology between DGAT1 and DGAT2. Both enzymes are widely expressed however some differences do exist in the relative abundance of expression in various tissues.

The gene encoding mouse DGAT1 has been used to create DGAT knock-out. These mice, although unable to express a functional DGAT enzyme (Dgat−/− mice), are viable and continue to synthesize triglycerides (see Smith et al, Nature Genetics (2000) 25, 87-90). This would suggest that multiple catalytic mechanisms contribute to triglyceride synthesis, such as DGAT2. An alternative pathway has also been shown to form triglycerides from two diacylglycerols by the action of diacylglycerol transacylase (see Lehner and Kuksis, Progress in Lipid Research (1996) 35,169-210).

Dgat−/− mice are resistant to diet-induced obesity and remain lean. When fed a high fat diet, Dgat−/− mice maintain weights comparable to mice fed a diet with regular fat content. Dgat−/− mice have lower tissue triglyceride levels. The resistance to weight gain seen in the knockout mice, which have a slightly higher food intake, is due to an increased energy expenditure and increased sensitivity to insulin and leptin (see Smith et al, Nature Genetics (2000) 25, 87-90, Chen and Farese, Trends in Cardiovascular Medicine (2000) 10,188-192, Chen and Farese, Current Opinions in Clinical Nutrition and Metabolic Care (2002) 5, 359-363 and Chen et al, Journal of Clinical Investigation (2002) 109,1049-1055). Dgat−/− mice have reduced rates of triglyceride absorption, improved triglyceride metabolism, and improved glucose metabolism, with lower glucose and insulin levels following a glucose load, in comparison to wild-type mice (see Buhman et al, Journal of Biological Chemistry (2002) 277, 25474-25479 and Chen and Farese, Trends in Cardiovascular Medicine (2000) 10,188-192).

Disorders or imbalances in triglyceride metabolism, both absorption as well as de novo synthesis, have been implicated in the pathogenesis of a variety of disease risks These include obesity, insulin resistance syndrome, type II diabetes, dyslipidemia, metabolic syndrome (syndrome X) and coronary heart disease (see Kahn, Nature Genetics (2000) 25, 6-7, Yanovski and Yanovski, New England Journal of Medicine (2002) 346, 591-602, Lewis et al, Endocrine Reviews (2002) 23, 201, Brazil, Nature Reviews Drug Discovery (2002) 1, 408, Malloy and Kane, Advances in Internal Medicine (2001) 47, 111, Subauste and Burant, Current Drug Targets—Immune, Endocrine & Metabolic Disorders (2003) 3, 263-270 and Yu and Ginsberg, Annals of Medicine (2004) 36, 252-261). Compounds that can decrease the synthesis of triglycerides from diacylglycerol by inhibiting or lowering the activity of the DGAT enzyme would be of value as therapeutic agents for the treatment diseases associated with abnormal metabolism of triglycerides.

Known inhibitors of DGAT include: dibenzoxazepinones (see Ramharack, et al, EP1219716 and Burrows et al, 26^{th} National Medicinal Chemistry Symposium (1998) poster C-22), substituted amino-pyrimidino-oxazines (see Fox et al, WO2004047755), chalcones such as xanthohumol (see Tabata et al, Phytochemistry (1997) 46, 683-687 and Casaschi et al, Journal of Nutrition (2004) 134, 1340-1346), substituted benzyl-phosphonates (see Kurogi et al, Journal of Medicinal Chemistry (1996) 39, 1433-1437, Goto, et al, Chemistry and Pharmaceutical Bulletin (1996) 44, 547-551, Ikeda, et al, Thirteenth International Symposium on Athersclerosis (2003), abstract 2P-0401, and Miyata, et al, JP 2004067635), aryl alkyl acid derivatives (see Smith et al, WO2004100881 and US20040224997), furan and thiophene derivatives (see WO2004022551), pyrrolo[1,2b]pyridazine derivatives (see Fox et al, WO2005103907), and substituted sulfonamides (see Budd Haeberlein and Buckett, WO20050442500).

Also known to be inhibitors of DGAT are: 2-bromo-palmitic acid (see Colman et al, Biochimica et Biophysica Acta (1992) 1125, 203-9), 2-bromo-octanoic acid (see Mayorek and Bar-Tana, Journal of Biological Chemistry (1985) 260, 6528-6532), roselipins (see Noriko et al, (Journal of Antibiotics (1999) 52, 815-826), amidepsin (see Tomoda et al, Journal of Antibiotics (1995) 48, 942-7), isochromophilone, prenylflavonoids (see Chung et al, Planta Medica (2004) 70, 258-260), polyacetylenes (see Lee et al, Planta Medica (2004) 70, 197-200), cochlioquinones (see Lee et al, Journal of Antibiotics (2003) 56, 967-969), tanshinones (see Ko et al, Archives of Pharmaceutical Research (2002) 25, 446-448), gemfibrozil (see Zhu et al, Atherosclerosis (2002) 164, 221-228), and substituted quinolones (see Ko, et al, Planta Medica (2002) 68, 1131-1133). Also known to be modulators of DGAT activity are antisense oligonucleotides (see Monia and Graham, US20040185559).

A need exits in the art, however, for additional DGAT inhibitors that have efficacy for the treatment of metabolic disorders such as, for example, obesity, type II diabetes mellitus and metabolic syndrome. Further, a need exists in the art for DGAT inhibitors having IC₅₀ values less than about 1 μM.

### SUMMARY OF THE INVENTION

In an embodiment of the present invention, provided is a compound of formula (I):

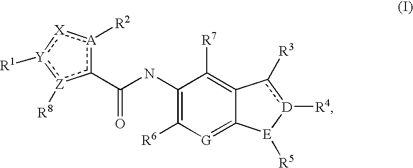

wherein:
R¹ is H, lower alkyl, cycloalkyl, heterocycloalkyl, aryl or heteroaryl;
R² is H, lower alkyl, haloloweralkyl or lower alkyl-alkoxy;
R³ is H, amino or ═O;
R⁴ is H or ═O or absent
R⁵ is lower alkyl, lower alkyl-alkoxy, alkoxyalkoxyalkyl, CH₂C(O)OCH₃, cycloalkyl, heterocycloalkyl, lower alkyl-hydroxy, aryl, heteroaryl, lower alkyl-aryl or lower alkyl-heteroaryl;
R⁶ is H, lower alkyl or alkoxy;
R⁷ is H, lower alkyl or alkoxy;
R⁸ is H, haloalkyl, lower alkyl or absent
X is S, C, O or N;
Y is C or N;
Z is C, N, O or S; and
A, D, E, G, independently of each other, is C or N,
   and pharmaceutically acceptable salts thereof.

In another embodiment of the present invention, provided is a pharmaceutical composition, comprising a therapeutically effective amount of a compound according to formula I or a pharmaceutically acceptable salt thereof and a pharmaceutically acceptable carrier.

### DETAILED DESCRIPTION OF THE INVENTION

The present invention pertains to DGAT inhibitors In a preferred embodiment, the invention provides compounds of the formula (I):

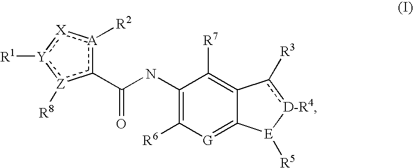

as well as pharmaceutically acceptable salts thereof.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent carbocyclic radical of three to seven, preferably three to six carbon atoms. This term is further exemplified by radicals such as cyclopropyl, cyclobutyl, cyclopentyl and cyclohexyl. In a preferred embodiment, the “cycloalkyl” moieties can optionally be substituted with one, two, three or four substituents, wherein each substituent is independently, for example, hydroxy, alkyl, alkoxy, halogen or amino, unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a cyclic alkyl ring, wherein one, two or three of the carbon ring atoms is replaced by a heteroatom such as N, O or S. Examples of heterocycloalkyl groups include, but are not limited to, morpholine, thiomorpholine, piperazine, piperidine and the like. The heterocycloalkyl groups may be unsubstituted or substituted.

The term “lower alkyl”, alone or in combination with other groups, refers to a branched or straight-chain monovalent alkyl radical of one to six carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic monovalent mono- or polycarbocyclic radical, such as phenyl or naphthyl, preferably phenyl.

The term “heteroaryl,” alone or in combination with other groups, means a monocyclic or bicyclic radical of 5 to 12 ring atoms having at least one aromatic ring containing one, two, or three ring heteroatoms selected from N, O, and S, the remaining ring atoms being C. One or two ring carbon atoms of the heteroaryl group may be replaced with a carbonyl group. The heteroaryl group described above may be substituted independently with one, two, or three substituents, preferably one or two substituents such as, for example, halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy and carboxyl C₁₋₆ alkoxy, preferably halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, carbamoyl, mono- or di-substituted amino-carbonyl, hydroxy-C₁₋₆ alkyl, carboxyl, C₁₋₆ alkoxy carbonyl and cyano.

The alkyl and aryl groups may be substituted or unsubstituted. Where substituted, there will generally be, for example, 1 to 3 substituents present, preferably 1 substituent. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono-or di-alkylaminocarbonylalkyl, arylaminocarbonyl), carbamates (e.g. alkoxycarbonylamino, arloxycarbonylamino, aminocarbonyloxy, mono-or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more, preferably one, heteroatom, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The lower alkyl groups may be substituted or unsubstituted, preferably unsubstituted. Where substituted, there will generally be, for example, 1 to 3 substituents present, preferably 1 substituent.

As used herein, the term “alkoxy” means alkyl-O—; and “alkoyl” means alkyl-CO—. Alkoxy substituent groups or alkoxy-containing substituent groups may be substituted by, for example, one or more alkyl groups.

As used herein, the term “halogen” means a fluorine, chlorine, bromine or iodine radical, preferably a fluorine, chlorine or bromine radical, and more preferably a fluorine or chlorine radical.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day

Compounds of the present invention can be prepared beginning with commercially available starting materials and utilizing general synthetic techniques and procedures known to those skilled in the art. Outlined below are reaction schemes suitable for preparing such compounds. Further exemplification is found in the specific examples listed below.

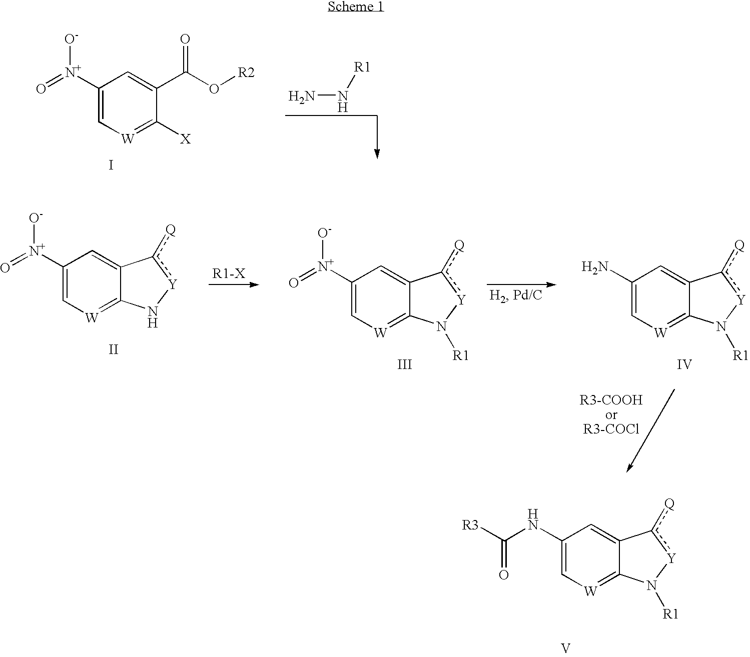

As shown in Scheme 1, compounds of the general type (II) (where Q is O, H or H2; Y is NH, CH, CH2 or C═O and W is CH or N) may be alkylated with an alkylating agent R1-X (where X is a halogen, and R1 is alkyl, arylalkyl, alkenyl or alkyloxyalkyl) in the presence of an organic or inorganic base to give III under conditions analogous to the ones described by Amrein et. al. in US 2006/0069269 A1 and Aran et. al. in Heterocycles 1997, 45, 129.

Alternatively, as shown in scheme 1, compounds of the general structure III (where W is either carbon or nitrogen, Y is NH and Q is O) may also be prepared by the condensation of a 2-halo-5-nitro ester I with a desirable hydrazine under conditions analogous to the ones described by Mitscher et. al. in Comb. Chem. High Throughput Screening 2003, 471. Hydrazines of the general structure H2NNHR1 are either commercially available or can be made in analogy to known literature procedures (such as J. Org. Chem. 1984, 49, 336, J. Med. Chem. 2004, 47, 2180, Bioorg. Med. Chem. 2004, 12, 1357 or Comb. Chem High Throughput Screening, 2003, 6, 471)

The reduction of aryl nitro compounds III to amines of the general structure IV can be done by Zn/NH4Cl or hydrogen in the presence of palladium on carbon or other suitable method known to those skilled in the art. Reaction of amine IV with an acid chloride R3COCl (where R3 is aryl, substituted aryl, heteroaryl, substituted heteroaryl, cycloalkyl or cycloheteroalkyl) in the presence of an organic or inorganic base results in amides of the general structure V. Alternatively, amine IV may be converted to amides of the general structure V by reaction with acid R3COOH (where R3 is aryl, substituted aryl, heteroaryl, substituted heteroaryl, cycloalkyl or cycloheteroalkyl) and an amide bond forming reagent such as BOP, PyBroP, EDCI or EDCI and HOBT often in the presence of a base such as triethyl amine under conditions analogous to the ones described in J. Chem. Soc. Perkin Trans. I 1025 (1985) or J. Org. Chem. 59 2437 (1994) or Int. J. Peptide Protein Res. 37 252 (1991).

As shown in scheme 2, a suitably substituted 5-nitro-2-halo aromatic cyanide VI (where Y is either nitrogen or carbon) may be condensed with an appropriate hydrazine to afford nitroaminoindazole VII. Cyanides of the general structure VI are either commercially available or they can be prepared in analogy to literature procedures (such as Synthetic Communications 2000, 30, 3047, Tetrahedron Lett. 1986, 27, 2203, or Synth. Commun. 1980, 47 2203). Hydrazines of the general structure H₂NNHR₂ are either commercially available or can be made in analogy to known literature procedures (such as J. Org. Chem. 1984, 49, 336, or J. Med. Chem. 2004, 47, 2180, or Bioorg. Med. Chem. 2004, 12, 1357 or Comb. Chem High Throughput Screening, 2003, 6, 471).

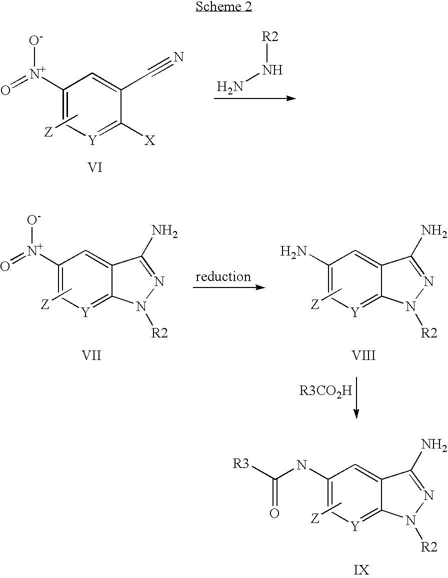

The reduction of nitro compounds VII to amines of the general structure VIII may be effected in an appropriate solvent with H₂ in the presence of palladium on carbon or with Zn/NH₄Cl or other suitable method known to persons skilled in the art. Amine VIII may be converted to amides of the general structure IX by reaction with an acid R3COOH (where R3 is aryl, substituted aryl, heteroaryl, substituted heteroaryl, cycloalkyl or cycloheteroalkyl) and an amide bond forming reagent such as BOP, PyBroP, EDCI or EDCI and HOBT often in the presence of a base such as triethyl amine under conditions analogous to the ones described in J. Chem. Soc. Perkin Trans. I 1025 (1985), J. Org. Chem. 59 2437 (1994), or Int. J. Peptide Protein Res. 37 252 (1991).

The invention will now be further described in the Examples below, which are intended as an illustration only and do not limit the scope of the invention.

### EXAMPLES

Definitions/List of Abbreviations
DGAT is diacylglycerol:acyl CoA O-acyltransferase
THF is tetrahydrofuran
DMF is N,N-dimethylformamide
DMA is N,N-dimethylacetamide
DMSO is dimethylsulfoxide
DCM is dichloromethane
DME is dimethoxyethane
MeOH is methanol
EtOH is ethanol
NBS is N-Bromosuccinimide
TFA is 1,1,1-trifluoroacetic acid
HOBT is 1-hydroxybenzotriazole
PyBroP is bromotripyrrolidinophosphonium hexafluorophosphate
EDCI is 1-[3-(dimethylamino)propyl]-3-ethylcarbodiimide hydrochloride
DIPEA is diisopropylethylamine
Brine is saturated aqueous solution of sodium chloride
DAG is 1,2-dioleoyl-sn-glycerol
TLC is thin layer chromatography
RP HPLC is reversed phase high performance liquid chromatography
HRMS is high resolution mass spectrometry
APCI-MS is atmospheric pressure chemical ionization mass spectrometry
ES-MS is electrospray mass spectrometry
LCMS is liquid chromatography mass spectrometry
RT is room or ambient temperature.

### Part I: Preparation of Preferred Intermediates

### Preparation of 1-ethyl-5-nitro-1,2-dihydro-indazol-3-one

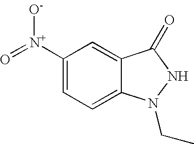

To a 20 mL reaction vial containing 2-fluoro-5-nitro-benzoic acid methyl ester and K₂CO₃ (919 mg, 3.33 mmol) in DMF (10 mL) was added ethyl hydrazine. The reaction was stirred at RT overnight and then partitioned between an aqueous citric acid solution (200 mL) and Et₂O (700 mL). The organic phase was washed with brine, dried over Na₂SO₄ and concentrated to dryness. The solid was triturated with CH₂Cl₂ and hexanes to afford the product, 1-ethyl-5-nitro-1,2-dihydro-indazol-3-one (420 mg, Yield: 61 %), LCMS calc. for C9H9N3O3 (m/e) 207, obsd. 208 (M+H)).

### Preparation of 1-methyl-5-nitro-1,2-dihydro-indazol-3-one

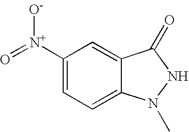

Methyl-5-nitro-1,2-dihydro-indazol-3-one was prepared from 2-fluoro-5-nitro-benzoic acid methyl ester and methyl hydrazine with a procedure similar to the one used in the preparation of 1-ethyl-5-nitro-1,2-dihydro-indazol-3-one (Yield: 14%). LCMS calcd for C8H7N3O3 (m/e) 193, obsd 194 (M+H).

### Preparation of 1-isopropyl-5-nitro-1,2-dihydro-indazol-3-one

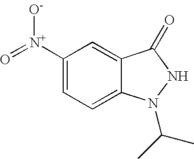

2-fluoro-5-nitro-benzoic acid methyl ester and K₂CO₃ (499 mg, 3.62 mmol) in DMF (10 mL) was added isopropylhydrazine and the reaction was stirred at 80° C. for 5 hr and then at 100° C. overnight. The reaction mixture was then cooled and partitioned between 0.1 M aqueous NaOH and EtOAc. The aqueous layer was acidified to pH 2 using conc. HCl and was extracted a second time with EtOAc. The resulting emulsion was treated with solid NaCl. The combined organic layer was then separated, washed with brine, dried over Na₂SO₄, and concentrated to dryness. Purification by flash chromatography using an Analogix instrument with an 80 g Redisep silica column and a 0-20% MeOH in CH₂Cl₂ gradient. The solid obtained from the purification was triturated with an Et₂O/CH₂Cl₂ mixture and hexanes to afford the product, 1-isopropyl-5-nitro-1,2-dihydro-indazol-3-one (80 mg, Yield: 25%), LCMS for C10H11N3O3 calc 221 (m/e), obsd. 222 (M+H)).

### Preparation of 5-nitro-1-propyl-1,2-dihydro-pyrazolo[3,4-b]pyridin-3-one

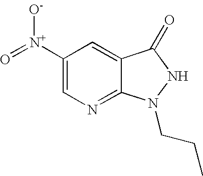

2-Chloro-5-nitro-nicotinic acid methyl ester (578 mg, 2.44 mmol) and n-propyl hydrazine oxalate (400 mg, 2.44 mmol) in DMF (10 ml) was treated with K₂CO₃ (672 mg, 4.87 mmol). The mixture was stirred at 80° C. for 3 hr and then at RT overnight. The reaction mixture was then concentrated and diluted with EtOAc. The organic layer was partitioned over water and solid citric acid was added to acidify the mixture to pH 1. The organic layer was washed with brine, dried over Na₂SO₄ and concentrated to dryness. The solid obtained was triturated with CH₂Cl₂ and then hexanes to afford the product, 5-nitro-1-propyl-1,2-dihydro-pyrazolo[3,4-b]pyridin-3-one (405 mg, Yield: 75%). LCMS calcd for C9H10N4O3 (m/e) 222, obsd. 223 (M+H).

### Preparation of 1-cyclohexyl-5-nitro-1,2-dihydro-indazol-3-one

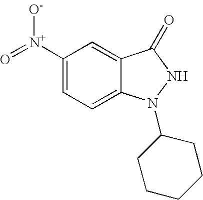

A mixture of 2-fluoro-5-nitro-benzoic acid methyl ester (715 mg, 3.32 mmol) and K₂CO₃ (916 mg, 6.64 mmol) in DMF (10 mL) was treated with cyclohexyl hydrazine HCl (500 mg, 3.32 mmol) (prepared as in J. Med. Chem. 2004, 12, 1357). The reaction was heated to 80° C. for 5 hr and then at 100° C. for 19 h and then at RT for 48 h. The reaction mixture was then partitioned between 0.1 M aqueous NaOH and EtOAc. The aqueous layer was acidified to pH 2 with aqueous conc. HCl and was extracted twice with EtOAc. The combined organic layer was washed with brine, dried over Na₂SO₄, and concentrated to dryness. The solid obtained was purified by flash chromatography using an Analogix instrument fitted with an 80 g Redisep silica gel column and a 0-20% MeOH in CH₂Cl₂ gradient to afford the product, 1-cyclohexyl-5-nitro-1,2-dihydro-indazol-3-one (124 mg, Yield: 14%). LCMS calcd for C13H15N3O3 (m/e) 261, obsd 262 (M+H).

### Preparation of 4-(5-nitro-3-oxo-2,3-dihydro-indazol-1-yl)-piperidine-1-carboxylic acid ethyl ester

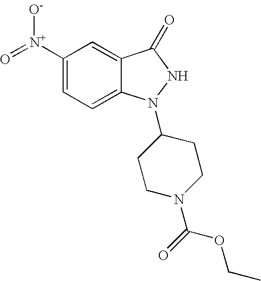

2-Fluoro-5-nitro-benzoic acid N-hydroxysuccinimide ester (prepared according to the general fashion described in Comb. Chem. High Throughput Screen. 2003, 6, 471-480) (241 mg, 0.85 mmol), in THF (5 mL), was cooled in an ice bath. Followed dropwise addition of a solution of 4-hydrazino-piperidine-1-carboxylic acid ethyl ester (167 mg, 0.89 mmol) (prepared as in J. Med. Chem. 2004, 47, 2180-2193) and DIEA (0.6 mL) in THF (3 mL). The reaction was stirred for 3 hr and then allowed to warm to room temperature. Followed solvent evaporation, dilution with EtOAc, and extraction with aqueous NaOH (1 M). The aqueous layer was acidified with concentrated HCl to pH 2 and extracted with EtOAc again. The combined organic layer was washed with brine, dried over Na₂SO₄, and concentrated. The residue was chromatographed on a silica gel column with a 0-100% of EtOAc in hexanes gradient. The intermediate acyl hydrazide obtained from this purification was immediately dissolved in DMSO and treated with N-trimethylsilyl-acetamide. After stirring at room temperature for 17 hr and for 2.5 hr at 110° C. in a sealed vessel, the mixture was cooled to room temperature and partitioned between EtOAc and aqueous NaOH (1M). The aqueous layer was acidified with aq. concentrated HCl to pH 2 and extracted with EtOAc again. The combined organic layer was washed with brine, dried over Na₂SO₄, and concentrated to the product, 4-(5-nitro-3-oxo-2,3-dihydro-indazol-1-yl)-piperidine-1-carboxylic acid ethyl ester (31 mg, 96% yield). LCMS for C15H18N4O5 calcd 334 (m/e), obsd 335 (M+H).

### Preparation of 1-benzyl-5-nitro-1,2-dihydro-indazol-3-one

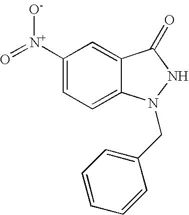

Benzyl bromide (10.5 g, 61 mmol) was added dropwise to a mixture of 5-nitro-1,2-dihydro-indazol-3-one (prepared as in Org. Synth. 1949, 29, 54 or Chem. Ber. 1942, 75, 1104) (10 g, 55.8 mmol) and NaOH (15%, 45 ml). The mixture was stirred at 80° C., for 1 h then cooled, neutralized with HCl (6N) and filtered. The solid obtained was washed with water and dried in airflow. The solid was then stirred in MeOH (25 ml) and ethyl acetate (25 ml) for 1 h then filtered and washed again with MeOH-ethyl acetate. After drying the solid was suspended in water (100 ml), NaOH (15%, 10 ml) was added and the mixture was stirred for 30 min. After filtration and a washing with water, the filtrate (mother liquid) was neutralized with HCl (1N) to pH=4-5. The product, 1-benzyl-5-nitro-1,2-dihydro-indazol-3-one was obtained by filtration (9.18 g, 61% yield). ES-MS calcd for C14H11N3O3 (m/e) 269, obsd 270 (M+H).

### Preparation of 5-nitro-1-pyridin-2-ylmethyl-1,2-dihydro-indazol-3-one

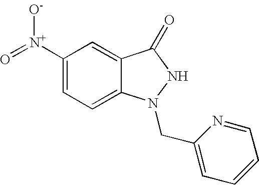

A mixture of 5-nitro-1,2-dihydro-indazol-3-one (306 mg, 1.71 mmol), 2-chloromethyl pyridine hydrochloride (281 mg, 1.71 mmol) and 1N aq. sodium hydroxide solution (5.13 mL, 5.13 mmol) in 2 ml dioxane was stirred at 70° C. for 3 hrs and then cooled. The purple reaction mixture was poured into H₂O and ethyl acetate. The pH of the solution was adjusted to 6 with aq. conc. HCl and the organic layer was separated. The aqueous layer was extracted with ethyl acetate (containing a small amount of MeOH). The combined organic layer was dried over MgSO₄, filtered and evaporated. The residue was purified by flash chromatography to afford the product 5-nitro-1-pyridin-2-ylmethyl-1,2-dihydro-indazol-3-one (316 mg, 68%). ES-MS calcd for C13H10N4O3 (m/e) 270.24, obsd 271.1 (M+H).

### Preparation of 1-(3-methoxy-benzyl)-5-nitro-1,2-dihydro-indazol-3-one

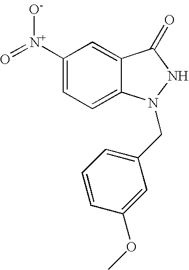

5-nitro-1,2-dihydro-indazol-3-one (250 mg, 1.27 mmol) and K₂CO₃ (180 mg, 1.27 mmol) in 6 mL of DMF was treated with 3-methoxy benzyl bromide (260 mg, 1.27 mmol) at room temperature. After stirring for 72 h the reaction mixture was partitioned between EtOAc and 1% aqueous citric acid. The pH of the aqueous layer was adjusted to 3 by adding solid citric acid monohydrate. The organic layer was then collected, dried over Na₂SO₄, filtered and concentrated. The residue was purified with a silica gel column and 40 to 100% EtOAc in hexanes to 30% THF in EtOAc gradient to afford the desired product 1-(3-methoxy-benzyl)-5-nitro-1,2-dihydro-indazol-3-one. (150 mg, 42%). HRMS for C15H13N3O4 (M+H) calcd: 300.0979. Found: 300.0979

### Preparation of 1-(4-methoxy-benzyl)-5-nitro-1,2-dihydro-indazol-3-one

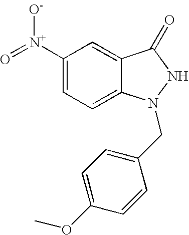

5-nitro-1,2-dihydro-indazol-3-one (200 mg, 1.12 mmol) and diisopropyl ethyl amine (0.24 mL, 1.34 mmol) in 10 mL of DMF was treated with 4-methoxy benzyl chloride (175 mg, 1.12 mmol) at room temperature. After stirring for 48 h the reaction mixture was partitioned between EtOAc and 1% aqueous citric acid. This resulted in a suspension in the organic phase. The organic phase was washed with H₂O and then evaporated to dryness. The solid was suspended in CH₂Cl₂ filtered and washed again with CH₂Cl₂ to afford the product 1-(4-methoxy-benzyl)-5-nitro-1,2-dihydro-indazol-3-one as a yellow solid (220 mg, 65%). HRMS for C15H13N3O4 (M+H) calcd: 300.0979. Found: 300.0979

### Preparation of 1-allyl-5-nitro-1,2-dihydro-indazol-3-one

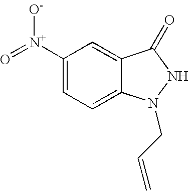

Starting from 5-nitro-1,2-dihydro-indazol-3-one and allyl bromide, 1-allyl-5-nitro-1,2-dihydro-indazol-3-one was prepared using a method similar to the one described in the synthesis of 1-benzyl-5-nitro-1,2-dihydro-indazol-3-one (67% yield). ES-MS calcd for C10H9N3O3 (m/e) 219, obsd 220 (M+H).

### Preparation of 1-allyl-5-nitro-1H-indole

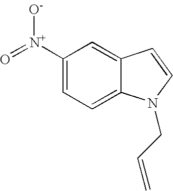

Starting from 5-nitro-1 H-indole and allyl bromide, 1-allyl-5-nitro-1H-indol was prepared using a method similar to the one described in the synthesis of 1-benzyl-5-nitro-1,2-dihydro-indazol-3-one. LCMS calcd for C11H10N2O2 (m/e) 202, obsd 203 (M+H).

### Preparation of 5-nitro-1-propyl-1H-pyrrolo[2,3-b]pyridine

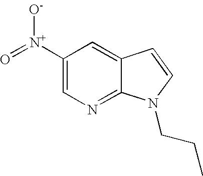

Starting from 5-nitro-1H-pyrrolo[2,3-b]pyridine and 1-iodo-propane, 5-nitro-1-propyl-1H-pyrrolo[2,3-b]pyridine was prepared using a method similar to the one described in the synthesis of 1-benzyl-5-nitro-1,2-dihydro-indazol-3-one. LCMS calcd for C10H11N3O2 (m/e) 205, obsd 206 (M+H).

### Preparation of 1-(2-methoxy-ethyl)-5-nitro-1H-indole

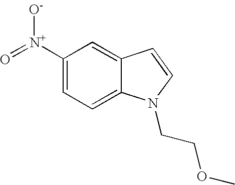

Starting from 5-nitro-1H-indole and 1-bromo-2-methoxy-ethane, 1-(2-methoxy-ethyl)-5-nitro-1H-indole was prepared using a method similar to the one described in the synthesis of 1-benzyl-5-nitro-1,2-dihydro-indazol-3-one. LCMS calcd for C11H12N2O3 (m/e) 220, obsd 221 (M+H).

### Preparation of 1-(2-methoxy-ethyl)-5-nitro-1H-indazole

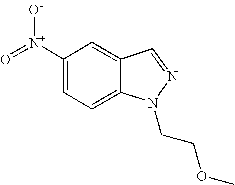

Starting from 5-nitro-1H-indazole and 1-chloro-2-methoxy-ethane, 1-(2-methoxy-ethyl)-5-nitro-1H-indazole was prepared using a method similar to the one described in the synthesis of 1-benzyl-5-nitro-1,2-dihydro-indazol-3-one. LCMS calcd for C10H11N3O3 (m/e) 221, obsd 222 (M+H).

### Preparation of 1-(2-methoxy-ethyl)-5-nitro-1H-pyrrolo[2,3-b]pyridine

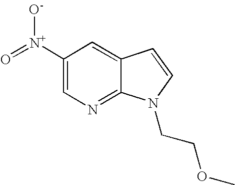

Starting from 5-nitro-1H-pyrrolo[2,3-b]pyridine and 1-chloro-2-methoxy-ethane, 1-(2-methoxy-ethyl)-5-nitro-1H-pyrrolo[2,3-b]pyridine was prepared using a method similar to the one described in the synthesis of 1-benzyl-5-nitro-1,2-dihydro-indazol-3-one. LCMS calcd for C10H11N3O3 (m/e) 221, obsd 222 (M+H).

### Preparation of 5-nitro-1-[2-(tetrahydro-pyran-2-yloxy)-ethyl]-1,2-dihydro-indazol-3-one

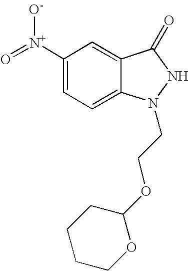

5-nitro-1,2-dihydro-indazol-3-one (200 mg, 1.12 mmol) and diisopropyl ethyl amine (0.29 mL, 1.68 mmol) in 6 mL of DMF was treated with 2-(2-bromoethoxy)tetrahydro-2H-pyran in a sealed vessel. The reaction mixture was heated at 80° C. for 48 h then cooled and partitioned between EtOAc and 1% aqueous citric acid (pH of aqueous layer 3). The organic layer collected, dried over Na₂SO₄, filtered and concentrated. The residue was chromatographed on a silica gel column using EtOAc to 5% EtOH in CH₂Cl₂ as elution system to afford the product 5-nitro-1-[2-(tetrahydro-pyran-2-yloxy)-ethyl]-1,2-dihydro-indazol-3-one (105 mg, 31%). HRMS for C14H17N3O5 [M+Na]⁺ calcd: 330.1060. Found: 330.1060.

### Preparation of (5-nitro-3-oxo-2,3-dihydro-indazol-1-yl)-acetic acid ethyl ester

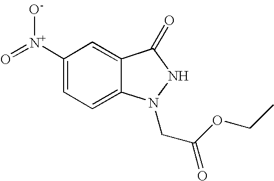

A mixture of 5-nitro-1,2-dihydro-indazol-3-one (0.5 g, 2.79 mmol), ethyl bromoacetate (309 μL, 2.79 mmol) and potassium carbonate (771 mg, 558 mmol) in DMF (5 mL) was stirred at room temperature overnight. The red reaction mixture was poured into 75 mL H₂O and 50 mL ethyl acetate. The pH of the solution was adjusted to 2 with HCl (conc) and the organic layer was separated. The aqueous layer was extracted with ethyl acetate. The combined organic layer was dried over MgSO₄, filtered and evaporated. The residue was purified by flash chromatography to afford the product (5-nitro-3-oxo-2,3-dihydro-indazol-1-yl)-acetic acid ethyl ester (0.38 g, 51%). ES-MS calcd for C11H11N3O5 (m/e) 265.2, obsd 264.1 (M−H).

### Preparation of (5-nitro-3-oxo-2,3-dihydro-indazol-1-yl)-acetic acid methyl ester

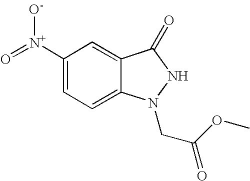

A mixture of 5-nitro-1,2-dihydro-indazol-3-one (500 mg, 2.79 mmol) and diisopropylethyl amine (1.5 mL) in DMF (15 mL) was treated with bromoacetic acid methyl ester (510 mg, 3.33 mmol). After stirring overnight at RT the reaction mixture was treated with solid citric acid monohydrate (3 g). After stirring for 10 minutes, EtOAc and H₂O were added. The organic layer was collected, dried over Na₂SO₄, filtered and concentrated. A silica gel column chromatography with 40-100% EtOAc in hexanes gradient followed by a trituration with CH₃CN afforded the product, 5-nitro-3-oxo-2,3-dihydro-indazol-1-yl)-acetic acid methyl ester (200 mg, 29%). HRMS for C10H9N3O5 [M+H]⁺ calcd: 252.0615. Found: 252.0615.

### Preparation of 1-[2-(2-methoxy-ethoxy)-ethyl]-5-nitro-1,2-dihydro-indazol-3-one

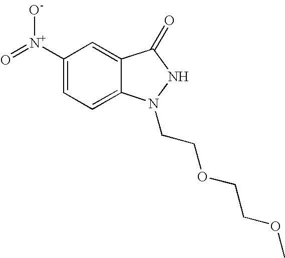

A mixture of 5-nitro-1,2-dihydro-indazol-3-one (399 mg, 2.22 mmol), 1-bromo-2-(2-methoxy-ethoxy)-ethane (454 uL, 3.34 mmol), potassium iodide (370 mg, 2.22 mmol) and 1N sodium hydroxide solution (6.7 mL, 6.7 mmol) in 2 ml dioxane was stirred at 60° C. overnight. The reaction mixture was then cooled, poured into 50 mL H₂O and 300 μL 10N NaOH was added. The aqueous layer was extracted with CH₂Cl₂ and then acidified to ˜pH 2 with 6N HCl. The aqueous layer was extracted with ethyl acetate. The combined organic layer was dried over MgSO₄, filtered and evaporated. The residue was purified by flash chromatography to afford the product 1-[2-(2-methoxy-ethoxy)-ethyl]-5-nitro-1,2-dihydro-indazol-3-one (380 mg, 61%). ES-MS calcd for C12H15N3O5 (m/e) 281.26, obsd 282.17 (M+H).

### Preparation of 1-(2-methoxy-ethyl)-5-nitro-1,2-dihydro-indazol-3-one

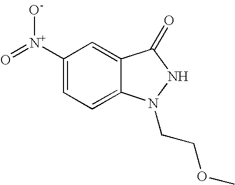

A mixture of 5-nitro-1,2-dihydro-indazol-3-one (402 mg, 2.24 mmol), 1-bromo-2-methoxy-ethane (332 μL, 3.53 mmol), potassium iodide (372 mg, 2.24 mmol) and 1N sodium hydroxide solution (6.7 mL, 6.7 mmol) in 2 ml dioxane was stirred at 60° C. for 12 hrs. The reaction mixture was then cooled and poured into 50 mL H₂O and 200 μL of 10N aq. NaOH was added. The aqueous layer was extracted with ether (30 mL) and CH₂Cl₂ (3×30 mL) and then acidified to pH 2 with 6N HCl. The aqueous layer was extracted with ethyl acetate (6×30 mL). The organic layers were combined, dried over MgSO₄, filtered and evaporated under vacuum to a yellow solid (430 mg). The crude product was purified by flash chromatography to yield 1-(2-methoxy-ethyl)-5-nitro-1,2-dihydro-indazol-3-one as a yellow solid (340 mg, Yield: 64%). ES-MS calcd for C10H11N3O4 (m/e) 237.21, obsd 238.0 (M+H).

### Preparation of 5-nitro-1-methyl-1H-indazol-3-ylamine

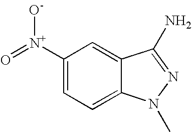

A solution of 2-fluoro-5-nitro-bezonitrile (300 mg, 1.81 mmol) and methyl hydrazine (170 mg, 3.62 mmol) in dioxane (10 mL) was stirred at 80° C. for 2 h. The reaction mixture was cooled and partitioned between 1N aqueous HCl and EtOAc. The organic layer was washed with water dried over Na₂SO₄ filtered and concentrated to the desired product 5-nitro-1-methyl-1H-indazol-3-ylamine (280 mg, 80%). HRMS for C8H8N4O2 (M+H) calcd: 193.0720. Found:193.0720.

### Preparation of 1-ethyl-5-nitro-1H-indazol-3-ylamine

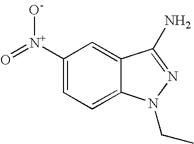

A mixture of 2-fluoro-5-nitrobenzonitrile (100 mg, 0.60 mmol) and ethylhydrazine oxalate (180 mg, 1.20 mmol) in DMF (10 mL) was treated with K₂CO₃ (500 mg, 3.62 mmol). The solution was heated at 80-90° C. until consumption of the limiting reagent then cooled and partitioned between EtOAc and 0.5 N aqueous HCl. The organic layer was washed with H₂O, dried over Na₂SO₄, filtered and concentrated to the product 1-ethyl-5-nitro-1H-indazol-3-ylamine (100 mg, 81%). HRMS for C9H10N4O2 (M+) calcd: 206.0804. Found: 206.0804.

### Preparation of 5-nitro-1-propyl-1H-indazol-3-ylamine

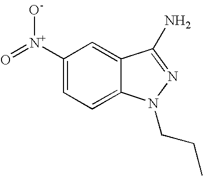

5-nitro-1-propyl-1H-indazol-3-ylamine was obtained from 2 fluoro-5-nitro-benzonitrile and propyl hydrazine oxalate following a procedure similar to the one described in the synthesis of 1-ethyl-5-nitro-1H-indazol-3-ylamine. The product, 5-nitro-1-propyl-1H-indazol-3-ylamine, was obtained after a precipitation out of CH₂Cl₂ with excess of hexanes (53% Yield). HRMS for C10H12N4O2 (M+H) calcd: 221.1033. Found: 221.1033.

### Preparation of 1-cyclohexyl-5-nitro-1H-indazol-3-ylamine

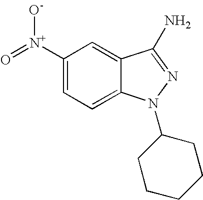

cyclohexyl-5-nitro-1H-indazol-3-ylamine was obtained from 2 fluoro-5-nitro-benzonitrile and cyclohexylhydrazine hydrochloride with a method similar to the one described in the synthesis of 1-ethyl-5-nitro-1H-indazol-3-ylamine. The product, 1-cyclohexyl-5-nitro-1H-indazol-3-ylamine, was obtained after silica gel column purification with 50% EtOAc in toluene as eluent (Yield: 19%). HRMS for C13H16N4O2 (M+H) calcd: 261.1346. Found: 261.1344.

### Preparation of 1-isopropyl-5-nitro-1H-indazol-3-ylamine

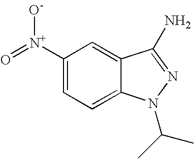

isopropyl-5-nitro-1H-indazol-3-ylamine was prepared from 2-fluoro-5-nitrobenzonitrile and isopropyl hydrazine hydrochloride with a method similar to the one described in the synthesis of 1-ethyl-5-nitro-1H-indazol-3-ylamine. The product, 1-isopropyl-5-nitro-1H-indazol-3-ylamine was obtained after silica gel column chromatography with a 0-50% EtOAc in hexanes gradient as the eluent (Yield: 38%). HRMS for C9H10N4O2 (M+) calcd: 220.0960. Found: 220.0960.

### Preparation of 1-(4-tert-butyl-phenyl)-5-nitro-1H-indazol-3-ylamine

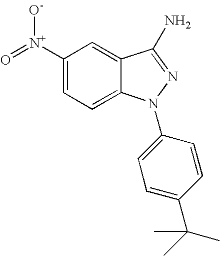

1-(4-tert-Butyl-phenyl)-5-nitro-1H-indazol-3-ylamine was prepared from 2-fluoro-5-nitrobenzonitrile (300 mg, 1.81 mmol) and 4-tert-butyl-phenyl hydrazine hydrochloride with a method similar to the one described in the synthesis of 5-nitro-1-phenyl-1H-indazol-3-ylamine. The product, 1-(4-tert-Butyl-phenyl)-5-nitro-1H-indazol-3-ylamine, was isolated after purification with an Isco CombiFlash Companion system fitted with a RediSep 80 g column and a 0-25% EtOAc in hexanes gradient (Yield: 5%). HRMS for C17H18N4O2 (M+H) calcd: 311.1503. Found: 311.1500.

### Preparation of 1-(3-tert-butyl-phenyl)-5-nitro-1H-indazol-3-ylamine

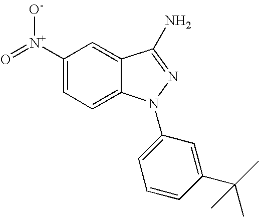

1-(3-tert-Butyl-phenyl)-5-nitro-1H-indazol-3-ylamine was prepared from 2-fluoro-5-nitrobenzonitrile and 3-tert-butyl-phenylhydrazine hydrochloride with a method similar to the one described in the synthesis of 5-nitro-1-phenyl-1H-indazol-3-ylamine. The product 1-(3-tert-Butyl-phenyl)-5-nitro-1H-indazol-3-ylamine was isolated after purification with an Isco CombiFlash Companion system fitted with a RediSep 80 g column and a 0-30% EtOAc in hexanes gradient as the eluent (Yield: 7%). HRMS for C17H18N4O2 (M+H) calcd: 311.1503. Found: 311.1501.

### Preparation of 4-chloro-1-ethyl-5-nitro-1H-indazol-3-ylamine

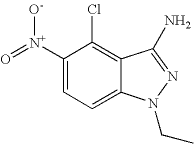

A mixture of 2-chloro-6-fluoro-3-nitrobenzonitrile (300 mg, 1.49 mmol) and ethylhydrazine oxalate (225 mg, 1.49 mmol) in DMF (20 mL) was treated with K₂CO₃ (500 mg, 3.62 mmol). The reaction mixture was then heated to 80° C. for 2 h then cooled and partitioned between EtOAc and 1N aqueous HCl. The organic layer was washed with water, dried over Na₂SO₄, filtered, evaporated and the resulting residue was passed through a pad of silica gel with 50% EtOAC in hexanes to afford the product 4-chloro-1-ethyl-5-nitro-1H-indazol-3ylamine (200 mg, 56%). HRMS for C9H9N4O2Cl (M+H) calcd: 241.0487. Found: 241.0486.

### Preparation of 1-ethyl-4-methoxy-5-nitro-1H-indazol-3-ylamine

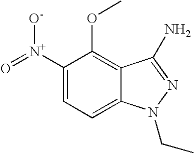

A solution of 4-chloro-1-ethyl-5-nitro-1H-indazol-3ylamine (105 mg, 0.44 mmol) in 5 mL of dry MeOH was treated with a 0.5 M solution of NaOMe in MeOH (1 mL). The solution was heated at 80° C. for 22 hr and then cooled and partitioned between EtOAc in 0.5 N aqueous HCl. The EtOAc layer was then washed with water and evaporated to afford the product, 1-ethyl-4-methoxy-5-nitro-1H-indazol-3-ylamine (80 mg, 78%). HRMS for C10H12N4O3 (M+H) calcd: 237.0982. Found: 237.0982.

### Preparation of 2-chloro-4-methoxy-5-nitro-benzoic acid

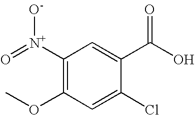

A mixture of 2-chloro-4-fluoro-5-nitro-benzoic acid (200 mg, 0.91 mmol), MeOH (5 mL) and DMF (0.2 mL) was treated with 0.5 M NaOMe in MeOH (3.64 mL, 1.82 mmol). The resulting mixture was heated for 3 h and then was allowed to stir at RT overnight. The mixture was then partitioned between EtOAc and 1N aqueous HCl. The organic layer was then collected, dried over Na₂SO₄ and evaporated to afford the product 2-chloro-4-methoxy-5-nitro-benzoic acid (200 mg, Yield: 95%). HRMS for C8H6NO5Cl (M+Na) calcd: 253.9827. Found: 253.9827.

### Preparation of 2-chloro-4-methoxy-5-nitro-benzamide

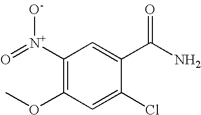

A suspension of 2-chloro-4-methoxy-5-nitro-benzoic acid (200 mg, 0.87 mmol) in CH₂Cl₂ (10 mL) was treated with oxallyl chloride (0.5 mL) and a catalytic amount of DMF. The suspension progressively went into solution. 15 min after complete dissolution was observed the reaction mixture was evaporated to a waxy solid that was then re-dissolved in CH₂Cl₂ (10 mL). The mixture was treated with a 7N solution of NH₃ in MeOH (0.3 mL, 2.17 mmol). After stirring for 5 min at RT the mixture was partitioned between EtOAc and H₂O. The EtOAc layer was then separated and evaporated to afford the desired product 2-chloro-4-methoxy-5-nitro-benzamide (187 mg, Yield: 93%). HRMS for C8H7ClN2O4 (M+) calcd: 230.0094. Found: 230.0097.

### Preparation of 2-chloro-4-methoxy-5-nitro-benzonitrile

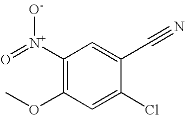

A solution of 2-chloro-4-methoxy-5-nitro-benzamide (179 mg, 0.78 mmol) in anhydrous dioxane (10 mL) was treated with diphosgene (160 μL). The reaction mixture was sealed and stirred at 70° C. for 3 h. The mixture was then cooled and the solvent was evaporated. The residue was purified by passing through a pad of silica gel using 40% EtOAc in hexanes as eluent to afford the product 2-chloro-4-methoxy-5-nitro-benzonitrile (95 mg, Yield: 57%). HRMS for C8H5N2O3Cl (M+) calcd: 211.9989. Found: 211.9986.

### Preparation of 1-ethyl-6-methoxy-5-nitro-1H-indazol-3-ylamine

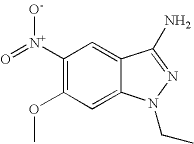

A mixture of 2-chloro-4-methoxy-5-nitro-benzonitrile (95 mg, 0.45 mmol) and ethylhydrazine oxalate (134 mg, 0.90 mmol) in DMF (10 mL) was treated with K₂CO₃ (500 mg, 3.62 mmol). The solution was heated at 100° C. for 2 h then cooled and partitioned between EtOAc and water. The water layer was acidified with conc. HCl to pH 4 and extracted thrice with EtOAc. The combined organic layer was concentrated and the residue was purified on a silica gel column with 40-100% EtOAc in hexanes gradient to afford the product 1-ethyl-6-methoxy-5-nitro-1H-indazol-3-ylamine (52 mg, Yield: 49%). HRMS for C10H12N4O3 (M+H) calcd: 237.0982. Found:237.0981.

### Preparation of 1-ethyl-5-nitro-1H-pyrazolo[3,4-b]pyridin-3-ylamine

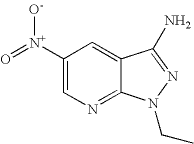

A mixture of 2-chloro-5-nitronicotinonitrile (200 mg, 1.09 mmol) and ethylhydrazine oxalate (490 mg, 3.27 mmol) in DMF (10 mL) was treated with K₂CO₃ (1.0 g, 7.24 mmol). The reaction mixture was heated at 80° C. for 1 h then cooled and partitioned between EtOAc and H₂O. The water layer was then acidified with aqueous conc. HCl to pH 6. The organic layer was collected and evaporated to afford the desired product 1-ethyl-5-nitro-1H-pyrazolo[3,4-b]pyridin-3-ylamine (200 mg, Yield: 88%). HRMS for C8H9N5O2 (M+H) calcd: 208.0829. Found: 208.0828.

### Preparation of 2-phenyl-5-bromomethyl -oxazole-4-carboxylic acid ethyl ester

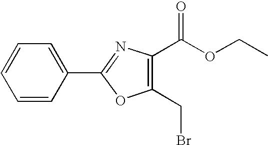

The 2-phenyl-5-methyl-oxazole-4-carboxylic acid (500 mg, 2.46 mmol) was mixed with CH₂Cl₂ (10 mL) and a catalytic amount of DMF. The mixture was cooled in an ice bath and oxalyl chloride (426 μL, 4.92) was added dropwise. The reaction was allowed to warm to RT over 2 h and was then concentrated to dryness. The acid chloride was dissolved in CH₂Cl₂ and dripped into EtOH (50 mL) that contained triethylamine (685 μL, 4.92 mmol). The solution stirred at RT for 2 hr, concentrated to a brown oil, dissolved in EtOAc and washed with 0.25 M aq. NaOH, 0.25 M aq. HCl and brine. The organic layer was dried over Na₂SO₄, concentrated and purified by flash chromatography using an Analogix system fitted with a 12 g RediSep silica gel column and a 0-20% EtOAc in hexanes gradient. The appropriate fractions were collected and concentrated to yield the product, 5-methyl-2-phenyl-oxazole-4-carboxylic ethyl ester (404 mg, Yield: 71%). LCMS calcd for C13H13NO3 (m/e) 231, obsd. 232 (M+H)).

The 5-methyl-2-phenyl-oxazole-4-carboxylic ethyl ester was then dissolved in CCl₄. NBS, and AIBN were added, and the mixture was refluxed for 2 h and then at RT overnight (17 h). Then the solvent was evaporated and the residue was supported on silica gel, and purified by flash chromatography using an Analogix system fitted with a 40 g Aspire column and a 50-100 CHCl₃ in hexane gradient. The appropriate fractions were combined concentrated and the residue was purified again using an Analogix system fitted with a 12 g RediSep column and a 0-20% EtOAc in hexanes gradient to afford the product, 2-phenyl-5-bromomethyl-oxazole-4-carboxylic acid ethyl ester (250 mg, Yield: 46%). LCMS for C13H12BrNO3, (m/e) calcd: 309. Obsd. 310 (M+H).

### Preparation of 1-pyridin-2-yl-3-trifluoromethyl-1H-pyrazole-4-carboxylic acid

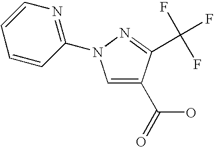

A mixture of 3-trifluoromethyl-1H-pyrazole-4-carboxylic acid ethyl ester (1.25 g, 6.0 mmol), copper (I) iodide (0.34 g, 1.8 mmol) and potassium carbonate (1.74 g, 12.6 mmol) in toluene (6 mL) in a round bottom flask was purged with argon. To the reaction mixture was then added 2-bromopyridine (0.69 mL, 7.2 mmol) and racemic trans-N,N′-dimethyl-cyclohexane-1,2-diamine (0.58 mL, 3.6 mmol). The slurry was heated under Ar in an oil bath at 110° C. for 24 hours. After cooling to room temperature, the reaction mixture was diluted with ethyl acetate and filtered over a bed of Celite®. The organic washings were combined and concentrated to give a crude which was purified by silica gel chromatography (Isco 120 g column, 0 to 40% ethyl acetate/hexanes) to give the intermediate 1-pyridin-2-yl-3-trifluoromethyl-1H-pyrazole-4-carboxylic acid ethyl ester (1.37 g, 80%).

A mixture of 1-pyridin-2-yl-3-trifluoromethyl-1H-pyrazole-4-carboxylic acid ethyl ester (1.35 g, 4.7 mmol) and 1N aqueous sodium hydroxide solution (23 mL) in methanol (20 mL) was stirred at room temperature overnight. The reaction mixture was acidified to pH˜4 with 1N aqueous hydrochloric acid. The mixture was then concentrated in vacuo to give 1-pyridin-2-yl-3-trifluoromethyl-1H-pyrazole-4-carboxylic acid, which was directly used in the next step without further purification. LCMS calcd for C10H6F3N3O2 (m/e) 257, obsd 258 (M+H).

### Preparation of 1-(4-fluoro-phenyl)-3-trifluoromethyl-1H-pyrazole-4-carboxylic acid

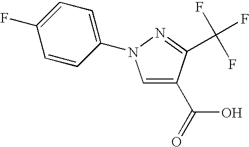

With a method similar to that used for the preparation of 1-pyridin-2-yl-3-trifluoromethyl-1H-pyrazole-4-carboxylic acid above, 1-(4-fluoro-phenyl)-3-trifluoromethyl-1H-pyrazole-4-carboxylic acid was prepared from 3-trifluoromethyl-1H-pyrazole-4-carboxylic acid ethyl ester and 1-fluoro-4-iodobenzene. LCMS calcd for C11H6F4N2O2 (m/e) 274, obsd 273 (M−H).

### Part II: Preparation of Preferred Compounds

### Example 1

### 2-Phenyl-5-trifluoromethyl-oxazole-4-carboxylic acid(3-oxo-1-propyl-2,3-dihydro-1H-indazol-5-yl)-amide

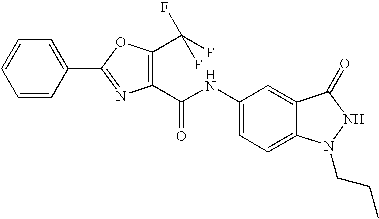

Allyl-5-nitro-1,2-dihydro-indazol-3-one (50 mg, 0.228 mmol) in 20 ml ethanol and 0.5 ml acetic acid was treated with 25 mg 10% Pd/C. The mixture was hydrogenated for 1.5 hrs under 20 psi hydrogen. The reaction mixture was filtered through a Celite® plug, evaporated and then re-evaporated from toluene and CH₃CN. The residue was dissolved in 2 ml THF and to this was added 2-phenyl-5-trifluoromethyl-oxazole-4-carboxylic acid (58.5 mg, 0.228 mmol), and triethylamine (35 μL, 0.250 mmol). The mixture was chilled in an ice bath and isobutyl chloroformate (32.8 μl, 0.250 mmol) in 2 mL of THF was added. The mixture was stirred for 30 minutes in the ice bath and then at room temperature overnight. Water and ethyl acetate were added to the mixture and the organic layer was separated. The ethyl acetate solution was extracted with saturated sodium bicarbonate, brine, dried over MgSO₄, filtered and evaporated to dryness. The residue was purified by flash chromatography to yield the product, 2-phenyl-5-trifluoromethyl-oxazole-4-carboxylic acid(3-oxo-1-propyl-2,3-dihydro-1H-indazol-5-yl)-amide (43 mg, Yield: 44%). ES-MS calcd for C21H17F3N4O3 (m/e) 431.4, obsd 472.3 (M+H+CH₃CN), 861.5 (2M+H).

### Example 2

### 1-Phenyl-5-trifluoromethyl-1H -pyrazole-4-carboxylic acid(3-oxo-1-propyl-2,3-dihydro-1H-indazol-5-yl)-amide

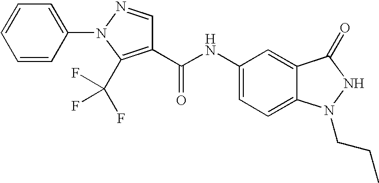

Allyl-5-nitro-1,2-dihydro-indazol-3-one (50 mg, 0.228 mmol) was hydrogenated as described above and treated with 1-phenyl-5-(trifluoromethyl)-1H-pyrazole-4-carboxylic acid (47.4 mg, 0.228 mmol), isobutylchloroformate (32.8 μL, 0.25 mmol) and triethylamine (38 μL, 0.27 mmol) as described above. The product, 1-phenyl-5-trifluoromethyl-1H-pyrazole-4-carboxylic acid(3-oxo-1-propyl-2,3-dihydro-1H-indazol-5-yl)-amide was isolated after flash column chromatography (11 mg, 11%). ES-MS calcd for C21H18F3N5O2 (m/e) 429.4, obsd 430.0 (M+H).

### Example 3

### 4-Methyl-2-phenyl-thiazole-5-carboxylic acid(3-oxo-1-propyl-2,3-dihydro-1H-indazol-5-yl)-amide

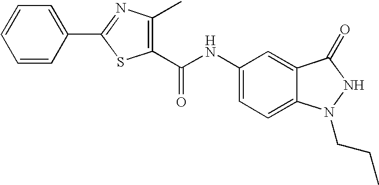

Allyl-5-nitro-1,2-dihydro-indazol-3-one (40 mg, 0.182 mmol) was hydrogenated under the conditions described in the synthesis of 2-phenyl-5-trifluoromethyl-oxazole-4-carboxylic acid(3-oxo-1-propyl-2,3-dihydro-1H-indazol-5-yl)-amide. The intermediate indazolone amine product was dissolved in 2 ml of CH₂Cl₂ and to this solution were added 4-methyl-2-phenyl-thiazole-5-carboxylic acid (40 mg, 0.182 mmol), BOP (160 mg, 0.364 mmol), triethylamine (128 μL, 0.182 mmol) and enough diisopropylethyl amine to keep the pH˜8. The reaction mixture was stirred until consumption of the starting materials. Water and EtOAc were then added to the mixture and the organic layer was separated. The ethyl acetate solution was extracted with saturated sodium bicarbonate, saturated sodium chloride, dried over MgSO₄, filtered and evaporated to dryness. The residue was purified by flash chromatography to yield 4-methyl-2-phenyl-thiazole-5-carboxylic acid(3-oxo-1-propyl-2,3-dihydro-1H-indazol-5-yl)-amide (Yield: 8%, 6 mg). ES-MS calcd for C21H20N4O2S (m/e) 392.5, obsd 393.3 (M+H).

### Example 4

### 2-Phenyl-5-trifluoromethyl-oxazole-4-carboxylic acid[1-(3-methoxy-benzyl)-3-oxo-2,3-dihydro-1H-indazol-5-yl]-amide

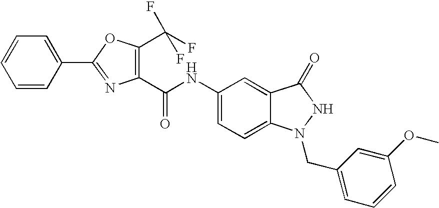

A solution of 1-(3-methoxy-benzyl)-5-nitro-1,2-dihydro-indazol-3-one (75 mg, 0.25 mmol) in EtOH was treated with 10% Pd/C (13 mg, 0.013 mmol) in EtOH (12 mL). The slurry was hydrogenated at atmospheric pressure until, as judged by TLC, consumption of the starting material was complete. The reaction mixture was then filtered, the solids were washed with EtOH and the combined organic layer was evaporated. The residue was dissolved in THF, followed addition of 2-phenyl-5-(trifluoromethyl)-oxazole-4-carboxylic acid (71 mg, 0.276 mmol) and EDCI (72 mg, 0.376 mmol). The resulting mixture was stirred at RT overnight and then partitioned between EtOAc and H₂O. The EtOAc layer was collected, dried over Na₂SO₄, filtered and concentrated. Chromatography with a silica gel column using a 40-100% EtOAc in hexanes to neat THF gradient followed by a second chromatography using a 20-100% CH₃CN in CH₂Cl₂ gradient afforded the product, 2-phenyl-5-trifluoromethyl-oxazole-4-carboxylic acid[1-(3-methoxy-benzyl)-3-oxo-2,3-dihydro-1H-indazol-5-yl]-amide, (45 mg, Yield: 35%). HRMS for C26H19N4O4F3 (M+H) calcd: 509.1431. Found: 509.1430

### Example 5

### 2-Phenyl-5-trifluoromethyl-oxazole-4-carboxylic acid(1-benzyl-3-oxo-2,3-dihydro-1H-indazol-5-yl)-amide

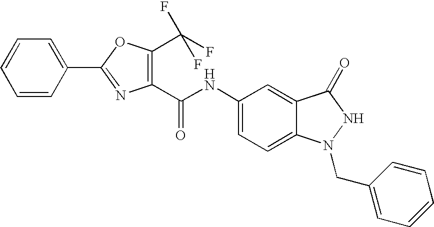

2-Phenyl-5-trifluoromethyl-oxazole-4-carboxylic acid(1-benzyl-3-oxo-2,3-dihydro-1H-indazol-5-yl)-amide was prepared from 2-phenyl-5-(trifluoromethyl)-oxazole-4-carboxylic acid and 1-benzyl-5-nitro-1,2-dihydro-indazol-3-one following a method similar to the one described in the synthesis of 2-phenyl-5-trifluoromethyl-oxazole-4-carboxylic acid [1-(3-methoxy-benzyl)-3-oxo-2,3-dihydro-1H-indazol-5-yl]-amide above. The crude reaction product was purified by silica gel column chromatography with neat EtOAc as the eluent (Yield: 35%). HRMS for C25H17N4O3F3 (M+H) calcd: 479.1236. Found: 479.1235.

### Example 6

### 2-Phenyl-5-trifluoromethyl-oxazole-4-carboxylic acid [1-(4-methoxy-benzyl)-3-oxo-2,3-dihydro-1H-indazol-5-yl]-amide

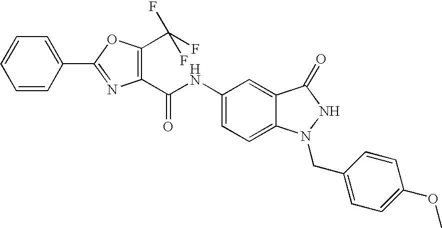

2-Phenyl-5-trifluoromethyl-oxazole-4-carboxylic acid[1-(4-methoxy-benzyl)-3-oxo-2,3-dihydro-1H-indazol-5-yl]-amide was prepared from 1-(4-methoxy-benzyl)-5-nitro-1,2-dihydro-indazol-3-one and 2-phenyl-5-(trifluoromethyl)-oxazole-4-carboxylic acid with a procedure similar to the one described in the synthesis of 2-phenyl-5-trifluoromethyl-oxazole-4-carboxylic acid[1-(3-methoxy-benzyl)-3-oxo-2,3-dihydro-1H-indazol-5-yl]-amide above. The crude reaction product was purified by silica gel column chromatography with EtOAc as the eluent (Yield: 30%). HRMS for C26H19N4O4F3 (M+H) calcd: 509.1431. Found: 509.1429.

### Example 7

### 2-Phenyl-5-trifluoromethyl-oxazole-4-carboxylic acid [1-(2-methoxy-ethyl)-3-oxo-2,3-dihydro-1H-indazol-5-yl]-amide

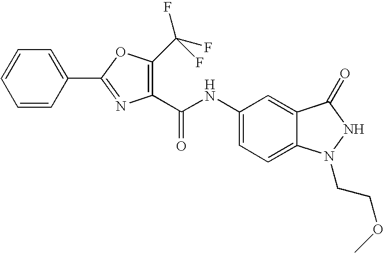

2-Phenyl-5-trifluoromethyl-oxazole-4-carboxylic acid[1-(2-methoxy-ethyl)-3-oxo-2,3-dihydro-1H-indazol-5-yl]-amide was prepared from 2-phenyl-5-(trifluoromethyl)-oxazole-4-carboxylic acid and 1-(2-methoxy-ethyl)-5-nitro-1,2-dihydro-indazol-3-one with a procedure similar to the one described in the synthesis of 2-phenyl-5-trifluoromethyl-oxazole-4-carboxylic acid[1-(3-methoxy-benzyl)-3-oxo-2,3-dihydro-1H-indazol-5-yl]-amide, above. The crude reaction product was purified by silica gel column chromatography with 50% CH₃CN in CH₂Cl₂ as the eluent (Yield: 44%). HRMS for C21H17F3N4O4 (M+H) calcd: 447.1275. Found: 447.1273.

### Example 8

### 5-Methyl-2-phenyl-2H-[1,2,3]triazole-4-carboxylic acid(1-ethyl-3-oxo-2,3-dihydro-1H-indazol-5-yl)-amide

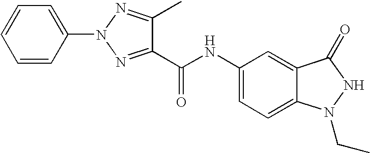

A solution of 1-ethyl-5-nitro-1,2-dihydro-indazol-3-one (100 mg, 0.483 mmol) in EtOH (20 mL) was treated with 10% Pd/C (50 mg, 0.048 mmol) and the slurry was hydrogenated under atmospheric pressure until, as judged by TLC, the consumption of the starting material was complete. The reaction mixture was then filtered through a pad of Celite®, the solids were washed with EtOH and then the combined organic layer was evaporated. The intermediate reduction product, 5-amino-1-ethyl-1,2-dihydro-indazol-3-one, was dissolved in DMF (10 mL), followed by addition of 5-methyl-2-phenyl-1H-[1,2,3]triazole-4-carboxylic acid (108 mg, 0.531 mmol) and EDCI (280 mg, 1.449 mmol). The mixture was stirred at RT overnight and then partitioned between EtOAc and H₂O. The organic layer was collected, dried over Na₂SO₄ filtered and concentrated. The residue was dissolved in boiling EtOAc and passed through a pad of silica gel using hot EtOAc. The product, 5-methyl-2-phenyl-2H-[1,2,3]triazole-4-carboxylic acid(1-ethyl-3-oxo-2,3-dihydro-1H-indazol-5-yl)-amide was finally isolated after a precipitation out of THF with excess of hexanes (50 mg, 28% yield). HRMS for C19H18N6O2 (M+H) calcd: 363.1564. Found: 363.1562

### Example 9

### 2-Phenyl-5-trifluoromethyl-oxazole-4-carboxylic acid(1-ethyl-3-oxo-2,3-dihydro-1H-indazol-5-yl)-amide

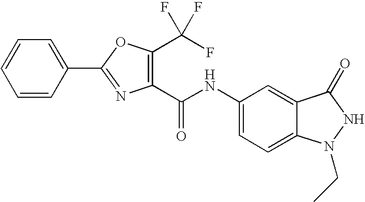

2-Phenyl-5-trifluoromethyl-oxazole-4-carboxylic acid(1-ethyl-3-oxo-2,3-dihydro-1H-indazol-5-yl)-amide was prepared from 1-ethyl-5-nitro-1,2-dihydro-indazol-3-one and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide, above. The crude reaction product was purified with flash column chromatography using a 0-5% MeOH in CH₂Cl₂ gradient (33 mg, yield 11%). LCMS calcd for C20H15F3N4O3 (m/e) 416, obsd. 417 (M+H).

### Example 10

### 2-(2-Trifluoromethoxy-phenyl)-5-trifluoromethyl-oxazole-4-carboxylic acid(1-ethyl-3-oxo-2,3-dihydro-1H-indazol-5-yl)-amide

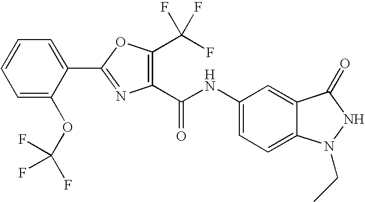

2-(2-Trifluoromethoxy-phenyl)-5-trifluoromethyl-oxazole-4-carboxylic acid (1-ethyl-3-oxo-2,3-dihydro-1H-indazol-5-yl)-amide was prepared from 1-ethyl-5-nitro-1,2-dihydro-indazol-3-one and 2-(2-trifluoromethoxy-phenyl)-5-trifluoromethyl-oxazole-4-carboxylic acid (prepared as in WO2007/060140 A2) with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide, above. The product was obtained after reverse phase HPLC purification (17 mg, yield 23%). LCMS calcd for C21H14F6N4O4 (m/e) 500, obsd. 501 (M+H).

### Example 11

### 4-Methyl-2-pyridin-2-yl-thiazole-5-carboxylic acid(1-ethyl-3-oxo-2,3-dihydro-1H-indazol-5-yl)-amide

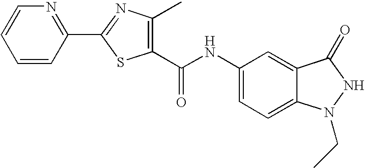

4-Methyl-2-pyridin-2-yl-thiazole-5-carboxylic acid(1-ethyl-3-oxo-2,3-dihydro-1H-indazol-5-yl)-amide was prepared from 1-ethyl-5-nitro-1,2-dihydro-indazol-3-one and 4-methyl-2-pyridin-2-yl-thiazole-5-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide, above. The product was obtained using flash column chromatography with a 0-20% THF in CH₂Cl₂ gradient and sequential triturations with MeOH, CH₂Cl₂ and hexanes (50 mg, yield: 15%). LCMS calcd for C19H17N5O2S (m/e) 379, obsd. 380 (M+H)).

### Example 12

### 5-Isopropyl-2-phenyl-oxazole-4-carboxylic acid(3-oxo-1-propyl-2,3-dihydro-1H-indazol-5-yl)-amide

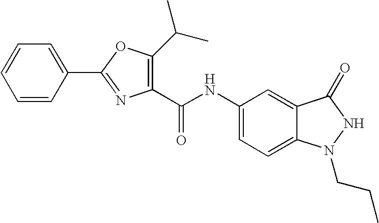

Starting from 5-isopropyl-2-phenyl-oxazole-4-carboxylic acid (prepared according to WO2007060140) and 1-allyl-5-nitro-1,2-dihydro-indazol-3-one, 5-isopropyl-2-phenyl-oxazole-4-carboxylic acid(3-oxo-1-propyl-2,3-dihydro-1H-indazol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C23H24N4O3 (m/e) 404, obsd 405 (M+H).

### Example 13

### 2-Phenyl-5-methoxymethyl-oxazole-4-carboxylic acid(1-ethyl-3-oxo-2,3-dihydro-1H-indazol-5-yl)-amide

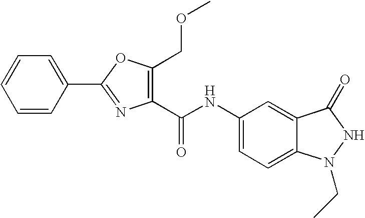

2-Phenyl-5-methoxymethyl-oxazole-4-carboxylic acid(1-ethyl-3-oxo-2,3-dihydro-1H-indazol-5-yl)-amide was prepared from 1-ethyl-5-nitro-1,2-dihydro-indazol-3-one and 5-methoxymethyl-2-phenyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide, above. After flash column chromatography with a 0-20% THF in CH₂Cl₂ gradient and sequential triturations with MeOH, CH₂Cl₂ and hexanes, the product was isolated (11 mg, yield: 17%). LCMS calcd for C21H20N4O4 (m/e) 392, obsd. 393 (M+H).

### Example 14

### 2-Phenyl-5-trifluoromethyl-oxazole-4-carboxylic acid{1-[2-(2-methoxy-ethoxy)ethyl]-3-oxo-2,3-dihydro-1H-indazol-5-yl}-amide

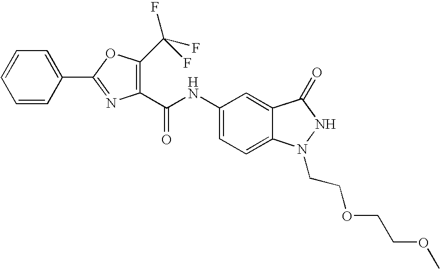

2-Phenyl-5-trifluoromethyl-oxazole-4-carboxylic acid{1-[2-(2-methoxy-ethoxy)ethyl]-3-oxo-2,3-dihydro-1H-indazol-5-yl}-amide was prepared from 2-phenyl-5-(trifluoromethyl)-oxazole-4-carboxylic acid and 1-[2-(2-methoxy-ethoxy)-ethyl]-5-nitro-1,2-dihydro-indazol-3-one with a procedure similar to the one described in the synthesis of 5-methyl-2-phenyl-2H-[1,2,3]triazole-4-carboxylic acid(1-ethyl-3-oxo-2,3-dihydro-1H-indazol-5-yl)-amide above. The crude reaction product was purified by silica gel column chromatography with EtOAc as the eluent (Yield: 14%). HRMS for C23H21F3N4O5 (M+H) calcd: 491.1537. Found: 491.1533

### Example 15

### {3-Oxo-5-[(2-phenyl-5-trifluoromethyl-oxazole-4-carbonyl)-amino]-2,3-dihydro-indazol-1-yl}-acetic acid methyl ester

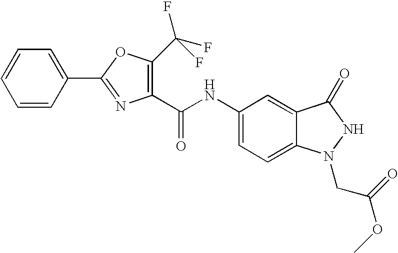

3-Oxo-5-[(2-phenyl-5-trifluoromethyl-oxazole-4-carbonyl)-amino]-2,3-dihydro-indazol-1-yl}-acetic acid methyl ester was prepared from 2-phenyl-5-(trifluoromethyl)-oxazole-4-carboxylic acid and (5-nitro-3-oxo-2,3-dihydro-indazol-1-yl)-acetic acid ethyl ester with a procedure similar to the one described in the synthesis of 5-methyl-2-phenyl-2H-[1,2,3]triazole-4-carboxylic acid(1-ethyl-3-oxo-2,3-dihydro-1H-indazol-5-yl)-amide above. The crude reaction product was purified by silica gel column chromatography with a 0-10% MeOH in EtOAc gradient as the eluent (Yield: 8%). HRMS for C21H15F3N4O5 (M+H) calcd: 461.1068. Found 461.1064.

### Example 16

### 2-Phenyl-5-trifluoromethyl-oxazole-4-carboxylic acid(1-methyl-3-oxo-2,3-dihydro-1H-indazol-5-yl)-amide

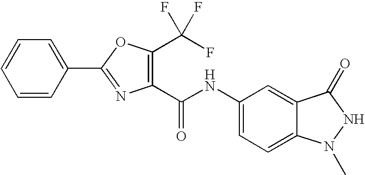

2-Phenyl-5-trifluoromethyl-oxazole-4-carboxylic acid(1-methyl-3-oxo-2,3-dihydro-1H-indazol-5-yl)-amide was prepared from 1-methyl-5-nitro-1,2-dihydro-indazol-3-one and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. Flash column chromatography with 5-100% MeOH in CH₂Cl₂ gradient followed by a recrystallization from MeOH afforded the product (Yield: 9%). LCMS calcd for C19H13F3N4O3 (m/e) 402, obsd. 403 (M+H)).

### Example 17

### 2-Phenyl-5-trifluoromethyl-oxazole-4-carboxylic acid (1-isopropyl-3-oxo-2,3-dihydro-1H-indazol-5-yl)-amide

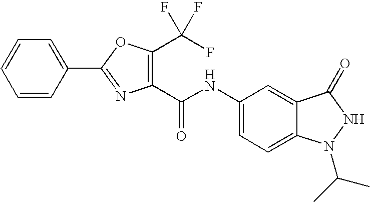

2-Phenyl-5-trifluoromethyl-oxazole-4-carboxylic acid (1-isopropyl-3-oxo-2,3-dihydro-1H-indazol-5-yl)-amide was prepared from 1-isopropyl-5-nitro-1,2-dihydro-indazol-3-one and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. The product was isolated after flash chromatography with a 0-3% MeOH in CH₂Cl₂ gradient (Yield 6%). LCMS calcd for C21H17F3N4O3 (m/e) 430, obsd 431 (M+H).

### Example 18

### 2-Phenyl-5-trifluoromethyl-oxazole-4-carboxylic acid (1-cyclohexyl-3-oxo-2,3-dihydro-1H-indazol-5-yl)-amide

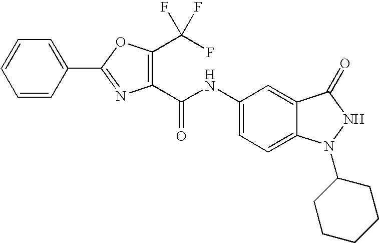

2-Phenyl-5-trifluoromethyl-oxazole-4-carboxylic acid (1-cyclohexyl-3-oxo-2,3-dihydro-1H-indazol-5-yl)-amide was prepared from 1-cyclohexyl-5-nitro-1,2-dihydro-indazol-3-one and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide above. The product was obtained after flash chromatography purification with a 0-5% MeOH in CH₂Cl₂ gradient (Yield: 5%). LCMS calcd for C24H21F3N4O3 (m/e) 470, obsd 471 (M+H).

### Example 19

### 4-{3-Oxo-5-[(2-phenyl-5-trifluoromethyl-oxazole-4-carbonyl)-amino]-2,3-dihydro-indazol-1-yl}-piperidine-1-carboxylic acid ethyl ester

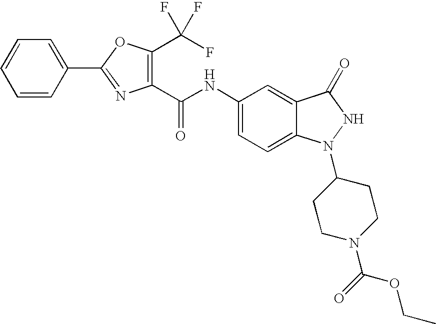

4-{3-Oxo-5-[(2-phenyl-5-trifluoromethyl-oxazole-4-carbonyl)-amino]-2,3-dihydro-indazol-1-yl}-piperidine-1-carboxylic acid ethyl ester was prepared from 4-(5-nitro-3-oxo-2,3-dihydro-indazol-1-yl)-piperidine-1-carboxylic acid ethyl ester and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide above. Flash column chromatography with a (0-30%) THF in CH₂Cl₂ gradient followed by preparative thin layer chromatography with 10% THF in CH₂Cl₂ afforded the product (Yield: 15%). LCMS calcd for C26H24F3N5O5 (m/e) 543, obsd 544 (M+H)).

### Example 20

### 2-Phenyl-5-trifluoromethyl-oxazole-4-carboxylic acid (3-oxo-1-propyl-2,3-dihydro-1H-pyrazolo[3,4-b]pyridin-5-yl)-amide

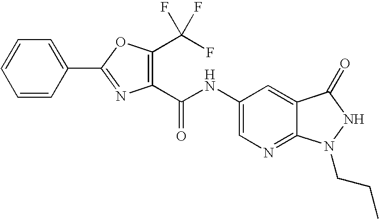

2-Phenyl-5-trifluoromethyl-oxazole-4-carboxylic acid(3-oxo-1-propyl-2,3-dihydro-1H-pyrazolo[3,4-b]pyridin-5-yl)-amide was prepared from 5-nitro-1-propyl-1,2-dihydro-pyrazolo[3,4-b]pyridin-3-one and 2-phenyl-5-trifluoromethyl-oxazole-4-carboxylic acid with a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide above. The product was obtained after flash column chromatography with a 0-3% MeOH in CH₂Cl₂ gradient (Yield: 14%). LCMS calcd for C20H16F3N5O3 (m/e) 431, obsd 432 (M+H)).

### Example 21

### 2-Phenyl-5-trifluoromethyl-oxazole-4-carboxylic acid(1-propyl-1H-indol-5-yl)-amide

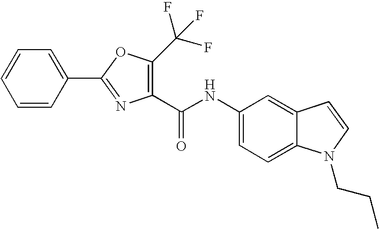

Starting from 2-phenyl-5-trifluoromethyl-oxazole-4-carboxylic acid and 1-allyl-5-nitro-1H-indole, 2-phenyl-5-trifluoromethyl-oxazole-4-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C22H18F3N3O2 (m/e) 413, obsd 414 (M+H).

### Example 22

### 5-Isopropyl-2-phenyl-oxazole-4-carboxylic acid(1-propyl-1H-indol-5-yl)-amide

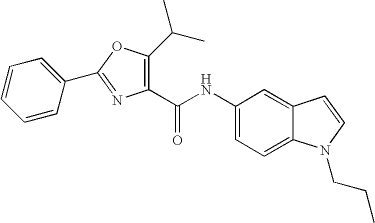

Starting from 5-isopropyl-2-phenyl-oxazole-4-carboxylic acid (prepared according to WO2007060140) and 1-allyl-5-nitro-1H-indole, 5-isopropyl-2-phenyl-oxazole-4-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C24H25N3O2 (m/e) 387, obsd 388 (M+H).

### Example 23

### 5-Ethyl-2-phenyl-oxazole-4-carboxylic acid(1-propyl-1H-indol-5-yl)-amide

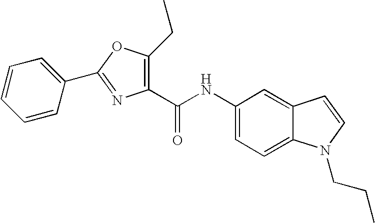

Starting from 5-ethyl-2-phenyl-oxazole-4-carboxylic acid (prepared according to WO 2007060140) and 1-allyl-5-nitro-1H-indole, 5-ethyl-2-phenyl-oxazole-4-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C23H23N3O2 (m/e) 373, obsd 374 (M+H).

### Example 24

### 4-Methyl-2-phenyl-thiazole-5-carboxylic acid(1-propyl-1H-indol-5-yl)-amide

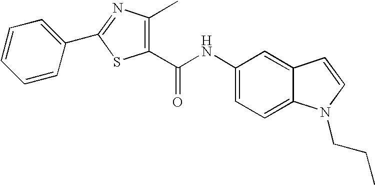

Starting from 4-methyl-2-phenyl-thiazole-5-carboxylic acid and 1-allyl-5-nitro-1H-indole, 4-methyl-2-phenyl-thiazole-5-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C22H21N3OS (m/e) 375, obsd 376 (M+H).

### Example 25

### 5-Methyl-2-phenyl-2H-[1,2,3]triazole-4-carboxylic acid(1-propyl-1H-indol-5-yl)-amide

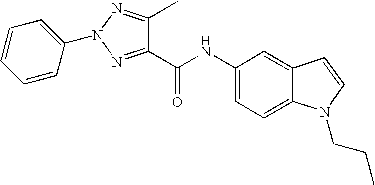

Starting from 5-methyl-2-phenyl-2H-[1,2,3]triazole-4-carboxylic acid and 1-allyl-5-nitro-1H-indole, 5-methyl-2-phenyl-2H-[1,2,3]triazole-4-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C21H21N5O (m/e) 359, obsd 360 (M+H).

### Example 26

### 2-Methyl-5-phenyl-furan-3-carboxylic acid(1-propyl-1H-indol-5-yl)-amide

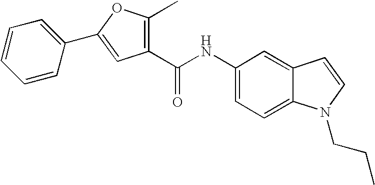

Starting from 2-methyl-5-phenyl-furan-3-carboxylic acid and 1-allyl-5-nitro-1H-indole, 2-methyl-5-phenyl-furan-3-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C23H22N2O2 (m/e) 358, obsd 359 (M+H).

### Example 27

### 5-Methyl-2-phenyl-oxazole-4-carboxylic acid(1-propyl-1H-indol-5-yl)-amide

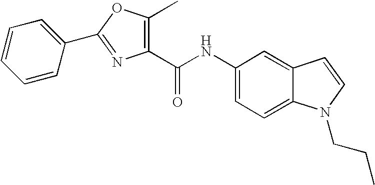

Starting from 5-methyl-2-phenyl-oxazole-4-carboxylic acid and 1-allyl-5-nitro-1H-indole, 5-methyl-2-phenyl-oxazole-4-carboxylic acid(1-propyl-1H-indol-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C22H21N3O2 (m/e) 359, obsd 360 (M+H).

### Example 28

### 2-Phenyl-5-trifluoromethyl-oxazole-4-carboxylic acid[1-(2-methoxy-ethyl)-1H-indol-5-yl]-amide

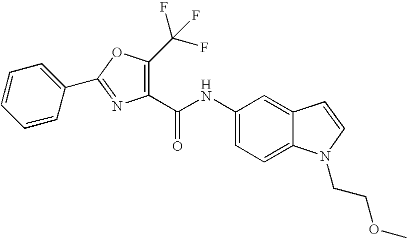

Starting from 2-phenyl-5-trifluoromethyl-oxazole-4-carboxylic acid and 1-(2-methoxy-ethyl)-5-nitro-1H-indole, 2-phenyl-5-trifluoromethyl-oxazole-4-carboxylic acid[1-(2-methoxy-ethyl)-1H-indol-5-yl]-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C22H18F3N3O3 (m/e) 429, obsd 430 (M+H).

### Example 29

### 2-Phenyl-5-trifluoromethyl-oxazole-4-carboxylic acid(1-propyl-1H-pyrrolo[2,3-b]pyridin-5-yl)-amide

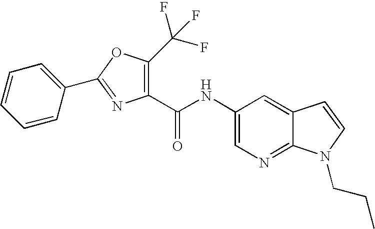

Starting from 2-phenyl-5-trifluoromethyl-oxazole-4-carboxylic acid and 5-nitro-1-propyl-1H-pyrrolo[2,3-b]pyridine, 2-phenyl-5-trifluoromethyl-oxazole-4-carboxylic acid(1-propyl-1H-pyrrolo[2,3-b]pyridin-5-yl)-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C21H17F3N4O2 (m/e) 414, obsd 415 (M+H).

### Example 30

### 2-Phenyl-5-trifluoromethyl-oxazole-4-carboxylic acid[1-(2-methoxy-ethyl)-1H-pyrrolo[2,3-b]pyridin-5-yl]-amide

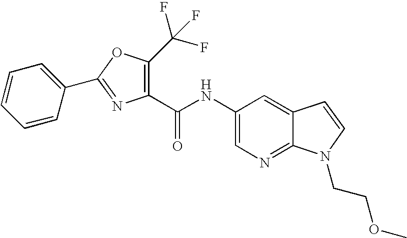

Starting from 2-phenyl-5-trifluoromethyl-oxazole-4-carboxylic acid and 1-(2-methoxy-ethyl)-5-nitro-1H-pyrrolo[2,3-b]pyridine, 2-phenyl-5-trifluoromethyl-oxazole-4-carboxylic acid[1-(2-methoxy-ethyl)-1H-pyrrolo[2,3-b]pyridin-5-yl]-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C21H17F3N4O3 (m/e) 430, obsd 431 (M+H).

### Example 31

### 2-Cyclohexyl-5-trifluoromethyl-oxazole-4-carboxylic acid[1-(2-methoxy-ethyl)-1H-pyrrolo[2,3-b]pyridin-5-yl]-amide

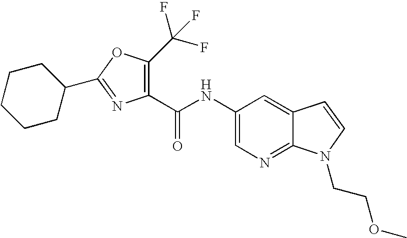

Starting from 2-cyclohexyl-5-trifluoromethyl-oxazole-4-carboxylic acid (prepared according to WO2007060140) and 1-(2-methoxy-ethyl)-5-nitro-1H-pyrrolo[2,3-b]pyridine, 2-cyclohexyl-5-trifluoromethyl-oxazole-4-carboxylic acid[1-(2-methoxy-ethyl)-1H-pyrrolo[2,3-b]pyridin-5-yl]-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C21H23F3N4O3 (m/e) 436, obsd 437 (M+H).

### Example 32

### 2-Phenyl-5-trifluoromethyl-oxazole-4-carboxylic acid [1-(2-methoxy-ethyl)-1H-indazol-5-yl]-amide

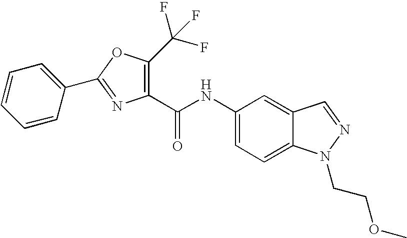

Starting from 2-phenyl-5-trifluoromethyl-oxazole-4-carboxylic acid and 1-(2-methoxy-ethyl)-5-nitro-1H-indazole, 2-phenyl-5-trifluoromethyl-oxazole-4-carboxylic acid[1-(2-methoxy-ethyl)-1H-indazol-5-yl]-amide was prepared using a method similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C21H17F3N4O3 (m/e) 430, obsd 431 (M+H).

### Example 33

### 2-Phenyl-5-trifluoromethyl-oxazole-4-carboxylic acid(1-propyl-2,3-dihydro-1H-indol-5-yl)-amide

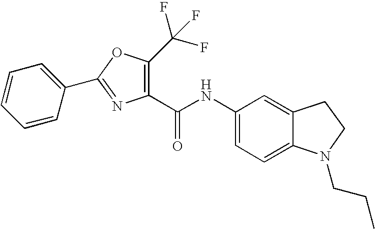

2-Phenyl-5-trifluoromethyl-oxazole-4-carboxylic acid(1-propyl-1H-indol-5-yl)-amide (50 mg, 0.12 mmol) was dissolved in 3 mL TFA and treated with excess sodium cyanoborohydride. The reaction was stirred at room temperature for 1 h. The reaction was then concentrated and the residue purified by HPLC to afford product, 2-phenyl-5-trifluoromethyl-oxazole-4-carboxylic acid(1-propyl-2,3-dihydro-1H-indol-5-yl)-amide (12 mg, 24% yield). LCMS calcd for C22H20F3N3O2 (m/e) 415, obsd 416 (M+H).

### Example 34

### 2-Phenyl-5-trifluoromethyl-oxazole-4-carboxylic acid(2,3-dioxo-1-propyl-2,3-dihydro-1H-indol-5-yl)-amide

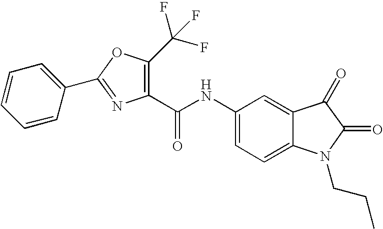

5-Nitro-1-propyl-1H-indole-2,3-dione (2.34 g, 10 mmol) was dissolved in 150 mL of MeOH, excess of zinc powder (1.6 g, 25 mmol) and solution of NH₄Cl (5.3 g, 100 mmol) in 70 mL of water were added to the reaction. The reaction was stirred at room temperature for 2 h. The reaction was filtered and the solution was concentrated. The dry residue was then mixed with water and extracted with EtOAc. The organic layers were combined, washed with brine, dried over on Na₂SO₄, and concentrated. The obtained 5-amino-1-propyl-1H-indole-2,3-dione was used directly in the following step without further purification. Starting from 2-phenyl-5-trifluoromethyl-oxazole-4-carboxylic acid and 5-amino-1-propyl-1H-indole-2,3-dione, 2-phenyl-5-trifluoromethyl-oxazole-4-carboxylic acid(2,3-dioxo-1-propyl-2,3-dihydro-1H-indol-5-yl)-amide was prepared using a standard coupling reaction similar to the one described in the synthesis of 5-methyl-2-phenyl-2H-[1,2,3]triazole-4-carboxylic acid(1-ethyl-3-oxo-2,3-dihydro-1H-indazol-5-yl)-amide. LCMS calcd for C22H16F3N3O4 (m/e) 443, obsd 444 (M+H).

### Example 35

### 1-Pyridin -2-yl-3-trifluoromethyl-1H -pyrazole-4-carboxylic acid(1-ethyl-3-oxo-2,3-dihydro-1H-indazol-5-yl)-amide

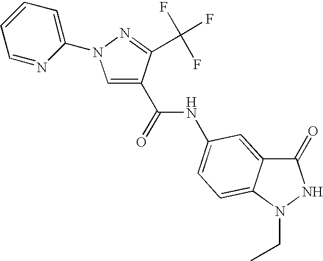

Pyridin-2-yl-3-trifluoromethyl-1H-pyrazole carboxylic acid (0.20 g, 0.77 mmol), the reduction product of 1-ethyl-5-nitro-1,2-dihydro-indazolone (5-amino-1-ethyl-1,2-dihydro-indazolone, obtained as described above in the synthesis of 5-methyl-2-phenyl-2H-[1,2,3]triazole-4-carboxylic acid(1-ethyl-3-oxo-2,3-dihydro-1H-indazol-5-yl)-amide) (0.13 g, 0.77 mmol), bromo-tris-pyrrolidino-phosphonium hexafluorophosphate (0.50 g 1.16 mmol) and diisopropylethylamine (0.40 mL, 2.3 mmol) in anhydrous dichloromethane (15 mL) was stirred at room temperature overnight. The organic layer was then evaporated. The residue was dissolved in methanol adsorbed onto silica gel and purified by silica gel chromatography (using ethyl acetate/hexanes as eluent) to give 1-pyrdin-2-yl-3-trifluoromethyl-1H-pyrazole-4-carboxylic acid(1-ethyl-3-oxo-2,3-dihydro-1H-indazol)-amide. LCMS calcd for C19H15F3N6O2 (m/e) 416, obsd 417 (M+H).

### Example 36

### 1-(4-Fluoro-phenyl)-3-trifluoromethyl-1H-pyrazole-4-carboxylic acid(1-ethyl-3-oxo-2,3-dihydro-1H-indazol-5-yl)-amide

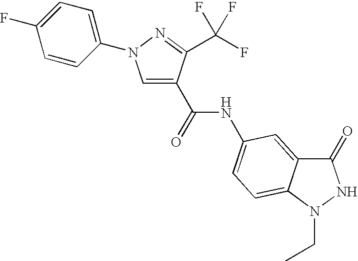

1-(4-fluoro-phenyl)-3-trifluoromethyl-1H-pyrazole-4-carboxylic acid(1-ethyl-3-oxo-2,3-dihydro-1H-indazol-5-yl)-amide was prepared from 1-(4-fluoro-phenyl)-3-trifluoromethyl-pyrazole-4-carboxylic acid and the reduction product of 1-ethyl-5-nitro-1,2-dihydro-indazol-3-one with a method similar to that used for the preparation of 1-pyridin-2-yl-3-trifluoromethyl-1H-pyrazole-4-carboxylic acid(1-ethyl-3-oxo-2,3-dihydro-1H-indazol-5-yl)-amide above. LCMS calcd for C20H15F4N5O2 (m/e) 433, obsd 434 (M+H).

### Example 37

### 2-Phenyl-5-trifluoromethyl-oxazole-4-carboxylic acid [1-(2-hydroxy-ethyl)-3-oxo2,3-dihydro-1H-indazol-5-yl]-amide

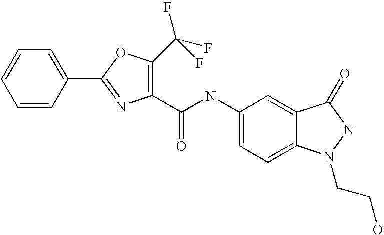

A solution of 5-nitro-1-[2-(tetrahydro-pyran-2-yloxy)-ethyl]-1,2-dihydro-indazol-3-one (100 mg, 0.325 mmol) in EtOH (10 mL) was treated with 10% Pd/C (17 mg, 0.016 mmol) and the mixture was then hydrogenated at atmospheric pressure for 2 h. The mixture was then filtered over a pad of Celite®. The solids were washed with ETOH and the combined filtrate was concentrated. The residue was dissolved in a 1:1 mixture of DMF and THF and to the resulting solution 2-phenyl-5-(trifluoromethyl)-oxazole-4-carboxylic acid (125 mg, 0.488 mmol) and EDCI (94 mg, 0.488 mmol) was added. After stirring overnight at room temperature the mixture was partitioned between EtOAc and H₂O. The organic layer was dried over Na₂SO₄, filtered and concentrated to a residue that was chromatographed on a silica gel column using 1-10% EtOH in CH₂Cl₂ gradient as elution system. The intermediate obtained from this purification was dissolved in methanol (15 mL) and the solution was treated with a catalytic amount of TsOH monohydrate. After stirring for 2 hrs at RT the reaction mixture was partitioned between EtOAc and H₂O. The EtOAc layer was dried over Na₂SO₄, filtered and concentrated to afford the product 2-phenyl-5-trifluoromethyl-oxazole-4-carboxylic acid[1-(2-hydroxy-ethyl)-3-oxo-2,3-dihydro-1H-indazol-5-yl]-amide (10 mg, 7% yield). HRMS cald for C20H15N4O4F3 433.1118, obsd 433.1116.

### General procedure for the preparation of pyrazole/triazole1-propyl substituted oxazolones of Examples 38 and 39 from a pyrazole/triazole acid chloride and 1-allyl-5-nitro-1,2-dihydro-indazol-3-one

A suspension of 1-allyl-5-nitro-1,2-dihydro-indazol-3-one (1 equv.) and Pd/C (10%, 3-5% equv.) in MeOH (25 ml/l mmol) was stirred under hydrogen atmosphere (balloon) at room temperature until completion of reduction. After removal of the catalyst the solution was evaporated. The residue was dissolved in acetonitrile, the solution formed was evaporated again and the resulting intermediate was then dried under high vacuum. The intermediate reduction product was then suspended in THF. The suspension was dispensed to vials (0.375 mmol each). In each vial triethylamine (3 eq.) was added and then followed addition of a pyrazole- or triazole-acyl chloride (0.25 M, 1 eq.). After shaking at room temperature overnight water (5 ml) was added to each reaction. Each reaction mixture was then extracted with ethyl acetate. Liquid-liquid handling was carried out on Tecan. Solvent removal followed by HPLC purification offered the pure compounds.

### Example 38

### 5-Methyl-2-phenyl-2H-[1,2,3]triazole-4-carboxylic acid(3-oxo-1-propyl-2,3-dihydro-H-indazol-5-yl)-amide

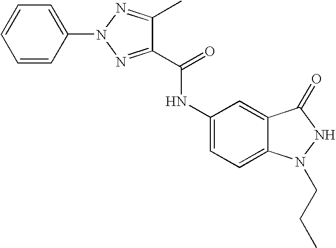

Following the general method described above, 5-methyl-2-phenyl-2H-[1,2,3]triazole-4-carboxylic acid(3-oxo-1-propyl-2,3-dihydro-H-indazol-5-yl)-amide was prepared from 1-allyl-5-nitro-1,2-dihydro-indazol-3-one and 5-methyl-2-phenyl-2H-1,2,3-triazole-4-carbonyl chloride (Yield: 59%) ES-MS calcd for C20H20N6O2 (m/e) 376.4, obsd 377.2 (M+H).

### Example 39

### 5-tert-Butyl-2-methyl-2H-pyrazole-3-carboxylic acid(3-oxo-1-propyl-2,3-dihydro-1H-indazol-5-yl)-amide

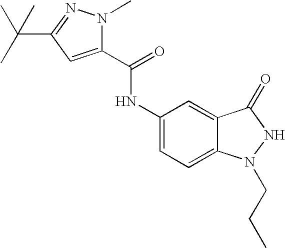

Following the general described above, 5-tert-butyl-2-methyl-2H-pyrazole-3-carboxylic acid(3-oxo-1-propyl-2,3-dihydro-1H-indazol-5-yl)-amide was prepared from 1-allyl-5-nitro-1,2-dihydro-indazol-3-one and 3-(tert-butyl)-1-methyl-1H-pyrazole-5-carbonyl chloride (Yield: 62%). ES-MS calcd for C19H25N5O2 (m/e) 355.4, obsd 356.2 (M+H).

### Example 40

### 2-Phenyl-5-trifluoromethyl-oxazole-4-carboxylic acid(3-amino-1-propyl-1H-indazol-5-yl)-amide

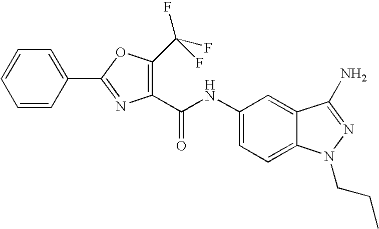

A solution of 5-nitro-1-propyl-1H-indazol-3-ylamine (150 mg, 0.68 mmol) in EtOH (20 mL was treated with 10% Pd/C (36 mg) and the resulting slurry was hydrogenated under 1 atm of hydrogen pressure until the consumption of the starting material, as judged by TLC, was complete. The mixture was then filtered through a pad of Celite®. The solids were washed with THF and the combined organic layer was evaporated to dryness. The residue was then dissolved in DMF (6 mL). Followed addition of 2-phenyl-5-(trifluoromethyl)-oxazole-4-carboxylic acid (210 mg, 0.82 mmol) and EDCI (390 mg, 2.04 mmol) and the reaction mixture was stirred at RT overnight and then partitioned between EtOAc and water. The organic layer was dried over Na₂SO₄, filtered and concentrated. Purification with a silica gel column using EtOAc as the eluent afforded the desired product, 2-phenyl-5-trifluoromethyl-oxazole-4-carboxylic acid(3-amino-1-propyl-1H-indazol-5-yl)-amide (80 mg, Yield: 27%). HRMS for C21H18F3N5O2 (M+H) calcd: 430.1486. Found: 430.1485

### Example 41

### 2-Phenyl-5-trifluoromethyl-oxazole-4-carboxylic acid(3-amino-1-methyl-1H-indazol-5-yl)-amide

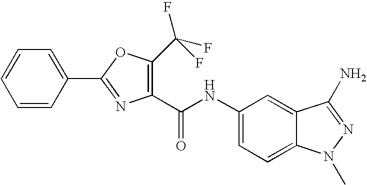

2-Phenyl-5-trifluoromethyl-oxazole-4-carboxylic acid(3-amino-1-methyl-1H-indazol-5-yl)-amide was prepared from 2-phenyl-5-(trifluoromethyl)-oxazole-4-carboxylic acid and 5-nitro-1-methyl-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above (Yield: 39%). HRMS for C19H14F3N5O2 (M+H) calcd: 402.1173. Found: 402.1173.

### Example 42

### 2-Phenyl-5-trifluoromethyl-oxazole-4-carboxylic acid(3-amino-1-cyclohexyl-1H-indazol-5-yl)-amide

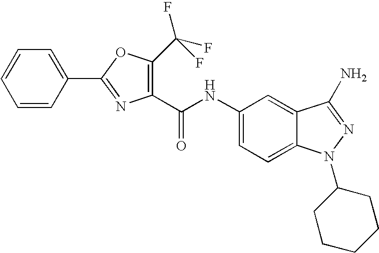

2-Phenyl-5-trifluoromethyl-oxazole-4-carboxylic acid(3-amino-1-cyclohexyl-1H-indazol-5-yl)-amide was prepared from 2-phenyl-5-(trifluoromethyl)-oxazole-4-carboxylic acid and 1-cyclohexyl-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified with a silica gel column using 40% EtOAc in hexanes as the eluent (Yield: 22%). HRMS for C24H22N5O2F3 (M+H) calcd: 470.1799. Found: 470.1793.

### Example 43

### 2-Phenyl-5-trifluoromethyl-oxazole-4-carboxylic acid(3-amino-1-ethyl-1H-indazol-5-yl)-amide

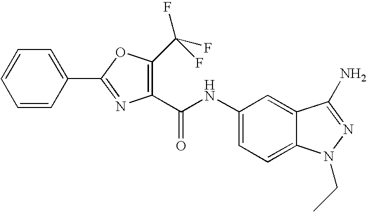

2-Phenyl-5-trifluoromethyl-oxazole-4-carboxylic acid(3-amino-1-ethyl-1H-indazol-5-yl)-amide was prepared from 2-phenyl-5-(trifluoromethyl)-oxazole-4-carboxylic acid and 1-ethyl-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The product was purified with a silica gel column and 50-100% EtOAc in hexanes gradient as the eluent (Yield: 37%). HRMS for C20H16F3N5O2 (M+H) calcd: 416.1329. Found: 416.1327.

### Example 44

### 2-Phenyl-5-trifluoromethyl-oxazole-4-carboxylic acid(3-amino-1-isopropyl-1H-indazol-5-yl)-amide

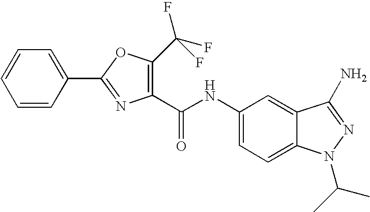

2-Phenyl-5-trifluoromethyl-oxazole-4-carboxylic acid(3-amino-1-isopropyl-1H-indazol-5-yl)-amide was prepared from 2-phenyl-5-(trifluoromethyl)-oxazole-4-carboxylic acid and 1-isopropyl-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The product was purified with a silica gel column using 50% Et₂O in toluene as the eluent (Yield: 25%). HRMS for C21H18F3N5O2 (M+H) calcd: 430.1486. Found: 430.1485.

### Example 45

### 2-Phenyl-5-trifluoromethyl-oxazole-4-carboxylic acid(3-amino-1-ethyl-4-methoxy-1H-indazol-5-yl)-amide

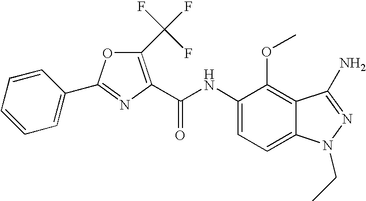

2-Phenyl-5-trifluoromethyl-oxazole-4-carboxylic acid(3-amino-1-ethyl-4-methoxy-1H-indazol-5-yl)-amide was prepared from 2-phenyl-5-(trifluoromethyl)-oxazole-4-carboxylic acid and 1-ethyl-4-methoxy-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified using a silica gel column and 40-100% EtOAc in hexanes gradient as the eluent (Yield: 36%). HRMS for C21H18F3N5O3 (M+H) calcd: 446.1435. Found: 446.1433.

### Example 46

### 2-Phenyl-5-trifluoromethyl-oxazole-4-carboxylic acid[3-amino-1-(4-tert-butyl-phenyl)-1H-indazol-5-yl]-amide

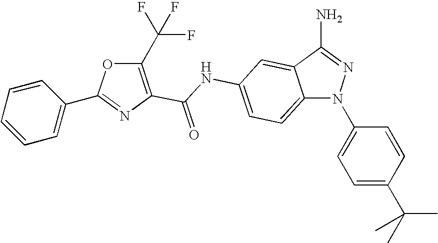

2-Phenyl-5-trifluoromethyl-oxazole-4-carboxylic acid [3-amino-1-(4-tert-butyl-phenyl)-1H-indazol-5-yl]-amide was prepared from 2-phenyl-5-(trifluoromethyl)-oxazole-4-carboxylic acid and 1-(4-tert-butyl-phenyl)-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product, was purified using an Isco CombiFlash Companion system fitted with a RediSep 40 g column using a 0-35% EtOAc in hexanes gradient as the eluent (Yield: 20%). HRMS for C28H24F3N5O2 (M+H) calcd: 520.1955. Found: 520.1954.

### Example 47

### 2-Phenyl-5-trifluoromethyl-oxazole-4-carboxylic acid[3-amino-1-(3-tert-butyl-phenyl)-1H-indazol-5-yl]-amide

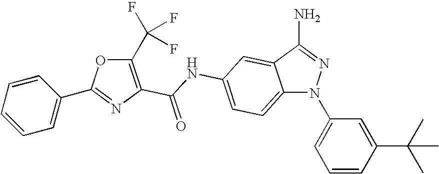

2-Phenyl-5-trifluoromethyl-oxazole-4-carboxylic acid [3-amino-1-(3-tert-butyl-phenyl)-1H-indazol-5-yl]-amide was prepared from 2-phenyl-5-(trifluoromethyl)-oxazole-4-carboxylic acid and 1-(3-tert-butyl-phenyl)-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified by an Isco CombiFlash Companion system fitted with a RediSep 40 g column using a 0-35% EtOAc in hexanes gradient as the eluent (Yield: 19%). HRMS for C28H24F3N5O2 (M+H) calcd: 520.1955. Found: 520.1957.

### Example 48

### 2-(2-Trifluoromethoxy-phenyl)-5-trifluoromethyl-oxazole-4-carboxylic acid(3-amino-1-ethyl-1H-indazol-5-yl)-amide

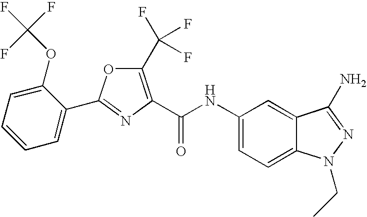

2-(2-Trifluoromethoxy-phenyl)-5-trifluoromethyl-oxazole-4-carboxylic acid (3-amino-1-ethyl-1H-indazol-5-yl)-amide was prepared from 2-(2-trifluoromethoxy-phenyl)-5-trifluoromethyl-oxazole-4-carboxylic acid (prepared as in WO2007/060140 A2) and 1-ethyl-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified by silica gel column using a 0-80% EtOAc in hexanes gradient as the eluent (Yield: 21%). HRMS for C21H15F6N5O3 (M+H) calcd: 500.1152. Found: 500.1147.

### Example 49

### 2-Phenyl-5-trifluoromethyl-oxazole-4-carboxylic acid(3-amino-1-ethyl-1H-pyrazolo[3,4-b]pyridin-5-yl)-amide

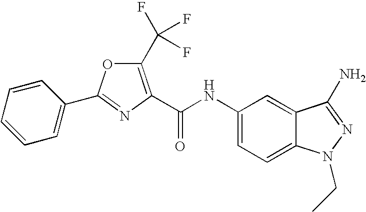

2-Phenyl-5-trifluoromethyl-oxazole-4-carboxylic acid(3-amino-1-ethyl-1H-pyrazolo[3,4-b]pyridin-5-yl)-amide was prepared from 2-phenyl-5-(trifluoromethyl)-oxazole-4-carboxylic acid and 1-ethyl-5-nitro-1H-pyrazolo[3,4-b]pyridin-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified by silica gel column chromatography using a 40-70% EtOAc in hexanes gradient as eluent (Yield: 59%). HRMS for C19H15N6O2F3 (M+H) calcd: 417.1282. Found: 417.1277.

### Example 50

### 2-Phenyl-5-trifluoromethyl-oxazole-4-carboxylic acid(3-amino-1-ethyl-6-methoxy-1H-indazol-5-yl)-amide

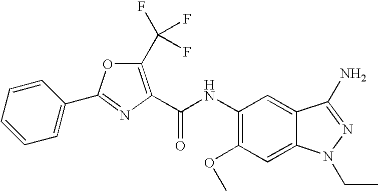

2-Phenyl-5-trifluoromethyl-oxazole-4-carboxylic acid(3-amino-1-ethyl-6-methoxy-1H-indazol-5-yl)-amide was prepared from 2-phenyl-5-(trifluoromethyl)-oxazole-4-carboxylic acid and 1-ethyl-6-methoxy-5-nitro-1H-indazol-3-ylamine using a procedure similar to the one described in the synthesis of 2-phenyl-5-trifluoromethyl-oxazole-4-carboxylic acid(3-amino-1-propyl-1H-indazol-5-yl)-amide above. The crude product was purified by silica gel column using a 50-100% EtOAc in hexanes gradient as eluent (Yield: 13%). HRMS for C21H18F3N5O3 (M+H) calcd: 446.1435. Found 446.1431

### Example 51

### DGAT Phospholipid FlashPlate Assay

Materials for the assay were: PL-FlashPlate: Phospholipid FlashPlates from PerkinElmer, catalog number SMP108; DAG (1,2-Dioleoyl-sn-glycerol) 10 mM suspended in water containing 0.1% Triton X-100; ¹⁴C-Pal-CoA (palmitoyl coenzyme A, [palmitoyl-1-¹⁴C]) from PerkinElmer, catalog number NEC-555 with a specific activity of 55 mCi/mmol; and DGAT pellet, with a protein concentration of 9.85 mg/ml.

Aqueous buffers were prepared or purchased as follows: The coating buffer (CB) was purchased from PerkinElmer, catalog number SMP900A; the reaction buffer (RB) was 50 mM Tris-HCl, pH 7.5, 100 mM NaCl, 0.01% BSA in water; the washing buffer (WB) is 50 mM Tris-HCl, pH 7.5, 100 mM NaCl, 0.05% deoxycholic acid sodium salt in water; the dilution buffer (DB) was 50 mM Tris-HCl, pH 7.5, 100 mM NaCl, 1 mM EDTA, 0.2% Triton X-100 in water.

1,2-Dioleoyl-sn-glycerol (DAG, 10 mmoles) was diluted to 500 μM with coating buffer (CB). The diluted DAG solution was then added to 384-well PL-FlashPlates at 60 μl per well, and incubated at room temperature for 2 days. The coated plates were then washed twice with washing buffer (WB) before use. Test compounds were serial diluted to 2000, 666.7, 222.2, 74.1, 24.7, 8.2, 2.7 and 0.9 μM in 100% DMSO. Diluted compound were further diluted 10 fold with reaction buffer (RB). ¹⁴C-Pal-CoA was diluted to 8.3 μM with RB. The DGAT pellet was diluted to 0.13 mg protein/ml with dilution buffer (DB) immediately before it was added to the PL-FlashPlates to start the reaction. 20 μl of the RB-diluted compounds (or 10% DMSO in RB for Total and Blank), 15 μl of RB diluted 14C-Pal-CoA and 15 μl of DB diluted DGAT pellet (DB without DGAT for Blanks) were transferred to each well of the PL-FlashPlates. The reaction mixtures were incubated at 37° C. for 1 hour. The reactions were stopped by washing 3 times with WB. Plates were sealed with Top-seal and read on a Topcount instrument.

Calculation of IC₅₀: The IC₅₀ value for each compound was generated using an Excel template. The Topcount rpm readings of Total and Blank were used as 0% and 100% inhibition. The percent inhibition values of reactions in the presence of compounds were calculated, and plotted against compound concentrations. All data were fitted into a Dose Response One Site model (4 parameter logistic model) as the following:

(A+((B−A)/(1+((x/C)̂D)))),

with A and B as the bottom and top of the curve (highest and lowest inhibition), respectively, and C as IC₅₀ and D as Hill Coefficient of the compound. The results are summarized in Table 1 below:

TABLE 1

| | Activity in DGAT Phospholipid FlashPlate |
|---|---|
| | Assay (A = IC₅₀ < 0.20 μM, |
| Compound in | B = IC₅₀ ≧ 0.20 μM) |
| Example 1 | A |
| Example 2 | B |
| Example 3 | A |
| Example 4 | B |
| Example 5 | A |
| Example 6 | B |
| Example 7 | A |
| Example 8 | A |
| Example 9 | A |
| Example 10 | A |
| Example 11 | B |
| Example 12 | B |
| Example 13 | B |
| Example 14 | B |
| Example 15 | A |
| Example 16 | B |
| Example 17 | A |
| Example 18 | B |
| Example 19 | A |
| Example 20 | B |
| Example 21 | B |
| Example 22 | B |
| Example 23 | B |
| Example 24 | B |
| Example 25 | B |
| Example 26 | B |
| Example 27 | B |
| Example 28 | B |
| Example 29 | B |
| Example 30 | B |
| Example 31 | B |
| Example 32 | B |
| Example 33 | B |
| Example 34 | B |
| Example 35 | A |
| Example 36 | B |
| Example 37 | B |
| Example 38 | A |
| Example 39 | B |
| Example 40 | B |
| Example 41 | B |
| Example 42 | B |
| Example 43 | A |
| Example 44 | A |
| Example 45 | B |
| Example 46 | B |
| Example 47 | B |
| Example 48 | B |
| Example 49 | B |
| Example 50 | B |

It is to be understood that the invention is not limited to the particular embodiments of the invention described above, as variations of the particular embodiments may be made and still fall within the scope of the appended claims.

What is claimed is:

1. A compound of formula (I): 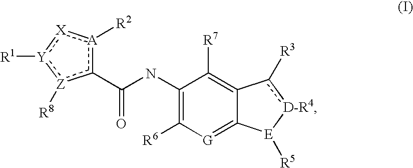
wherein:
R¹ is H, lower alkyl, cycloalkyl, heterocycloalkyl, aryl or heteroaryl;
R² is H, lower alkyl, haloloweralkyl or lower alkyl-alkoxy;
R³ is H, amino or ═O;
R⁴ is H or ═O or absent
R⁵ is lower alkyl, lower alkyl-alkoxy, alkoxyalkoxyalkyl, CH₂C(O)OCH₃, cycloalkyl,heterocycloalkyl, lower alkyl-hydroxy, aryl, heteroaryl, lower alkyl-aryl or lower alkyl-heteroaryl;
R⁶ is H, lower alkyl or alkoxy;
R⁷ is H, lower alkyl or alkoxy;
R⁸ is H, lower alkyl, haloalkyl or absent;
X is S, C, O or N;
Y is C or N;
Z is C, N, O or S; and
A, D, E, G, independently of each other, is C or N,
and pharmaceutically acceptable salts thereof.

2. The compound according to claim 1, wherein:
R¹ is lower alkyl, cycloalkyl or heterocycloalkyl; and
R⁵ is aryl, heteroaryl, lower alkyl-aryl or lower alkyl-heteroaryl.

3. The compound according to claim 1, wherein:
R¹ is lower alkyl, cycloalkyl or heterocycloalkyl; and
R⁵ is lower alkyl, lower alkyl-alkoxy, alkoxyalkoxyalkyl, CH₂C(O)OCH₃, cycloalkyl, heterocycloalkyl, lower alkyl-hydroxy.

4. The compound according to claim 1, wherein:
R¹ is aryl or heteroaryl; and
R⁵ is lower alkyl, lower alkyl-alkoxy, alkoxyalkoxyalkyl, CH₂C(O)OCH₃, cycloalkyl, heterocycloalkyl or lower alkyl-hydroxy.

5. The compound according to claim 1, wherein:
R¹ is aryl or heteroaryl; and
R⁵ is aryl, heteroaryl, lower alkyl-aryl or lower alkyl-heteroaryl.

6. The compound according to claim 1, wherein X is O and Z is N.

7. The compound according to claim 1, wherein D is N and E is N.

8. The compound according to claim 1, wherein X, Y and Z are N.

9. The compound according to claim 1, wherein X is N and Z is S.

10. The compound according to claim 1, wherein D is C and E is N

11. The compound according to claim 1, wherein R¹ is t-butyl, cyclohexyl, pyridyl or phenyl, unsubstituted or substituted with halogen, or haloloweralkoxy

12. The compound according to claim 1, wherein R² is a methyl, ethyl, isopropyl, trifluoromethyl or methoxymethyl group.

13. The compound according to claim 1, wherein R³ is amino.

14. The compound according to claim 1, wherein R⁴ is ═O.

15. The compound according to claim 1, wherein R⁵ is methyl, ethyl, propyl, isopropyl, methoxy-ethyl, hydroxy-ethyl, methoxy-ethoxy ethyl, acetic acid methyl ester, cyclohexyl, phenyl, unsubstituted or substituted with lower alkyl, CH₂-phenyl, unsubstituted or substituted with alkoxy, or piperidine, unsubstituted or substituted with C(O)OCH₂CH₃.

16. The compound according to claim 1, wherein R⁶ is a methoxy group.

17. The compound according to claim 1, wherein R⁷ is a methoxy group.

18. The compound according to claim 1, wherein R⁸ is a trifluoromethyl group.

18. The compound according to claim 1, wherein X is O.

19. The compound according to claim 1, wherein Y is C.

20. The compound according to claim 1, wherein Z is N.

21. The compound according to claim 1, wherein D is N.

22. The compound according to claim 1, wherein E is N.

23. The compound according to claim 1, wherein G is C.

25. The compound according to claim 1, wherein said compound is:
2-Phenyl-5-trifluoromethyl-oxazole-4-carboxylic acid(1-ethyl-3-oxo-2,3-dihydro-1H-indazol-5-yl)-amide,
5-Methyl-2-phenyl-2H-[1,2,3]triazole-4-carboxylic acid(3-oxo-1-propyl-2,3-dihydro-H-indazol-5-yl)-amide,
2-Phenyl-5-trifluoromethyl-oxazole-4-carboxylic acid(1-isopropyl-3-oxo-2,3-dihydro-1H-indazol-5-yl)-amide,
1-Pyridin-2-yl-3-trifluoromethyl-1H-pyrazole-4-carboxylic acid(1-ethyl-3-oxo-2,3-dihydro-1H-indazol-5-yl)-amide,
4-Methyl-2-phenyl-thiazole-5-carboxylic acid(3-oxo-1-propyl-2,3-dihydro-1H-indazol-5-yl)-amide,
2-Phenyl-5-trifluoromethyl-oxazole-4-carboxylic acid(3-oxo-1-propyl-2,3-dihydro-1H-indazol-5-yl)-amide,
2-Phenyl-5-trifluoromethyl-oxazole-4-carboxylic acid(3-amino-1-isopropyl-1H-indazol-5-yl)-amide,
{3-Oxo-5-[(2-phenyl-5-trifluoromethyl-oxazole-4-carbonyl)-amino]-2,3-dihydro-indazol-1-yl}-acetic acid methyl ester,
2-Phenyl-5-trifluoromethyl-oxazole-4-carboxylic acid [1-(2-methoxy-ethyl)-3-oxo-2,3-dihydro-1H-indazol-5-yl]-amide or
2- (2-Trifluoromethoxy-phenyl)-5-trifluoromethyl-oxazole-4-carboxylic acid(1-ethyl-3-oxo-2,3-dihydro-1H-indazol-5-yl)-amide.
